SEQUENCE LISTING (3 sequences)

SEQ ID NO: 1 - protein, 43 aa, Artificial Sequence
  Amyloid beta fragment
DAEFRHDSGYEVHHQKLVFFAEDVGSNKGAIIGLMVGGVVIAT

SEQ ID NO: 2 - DNA, 24 bp, Artificial Sequence
  mouse FPR2 primer
tctaccatctccagagttctgtgg

SEQ ID NO: 3 - DNA, 24 bp, Artificial Sequence
  mouse FPR2 primer
ttacatctaccacaatgtgaacta

(19) United States
(12) Patent Application Publication
(10) Pub. No.: US 2005/0130890 A1
(43) Pub. Date: Jun. 16, 2005

(54) Amyloid beta is a ligand for FPR class receptors

(76) Inventors: Ji Ming Wang, Frederick, MD (US); Yingying Le, Shanghai (CN); WangHua Gong, Frederick, MD (US); Hiroshi Yazawa, Frederick, MD (US); Zu-Xi Yu, Gaithersburg, MD (US); Joost J. Oppenheim, Bethesda, MD (US); Philip M. Murphy, Rockville, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 10/831,524

(22) Filed: Apr. 23, 2004

Related U.S. Application Data
(63) Continuation of application No. PCT/US02/34455, filed on Oct. 25, 2002.
(60) Provisional application No. 60/345,873, filed on Oct. 26, 2001.

(51) Int. Cl. G01N 33/53; G01N 33/567; A61K 38/17; C07K 14/705

(52) U.S. Cl. 514/12; 530/350; 435/7.2

(57) ABSTRACT

The invention relates to the discovery that amyloid β is a ligand for FPR class receptors, which mediate the inflammation associated with Alzheimer's disease.

### RELATED APPLICATIONS

This application is a continuation and claims the benefit of priority of International Application No. PCT/US02/34455 filed 25 Oct. 2002, designating the United States of America and published in English as WO 03/035006 on 1 May 2003, which claims the benefit of priority of U.S. Provisional Application No. 60/345,873 filed 26 Oct. 2001, both of which are hereby expressly incorporated by reference in their entireties.

### FIELD OF THE INVENTION

The invention relates to the discovery that amyloid β is a ligand for FPR(N-formylpeptide receptor) class receptors, which mediate inflammation associated with Alzheimer's disease.

### BACKGROUND OF THE INVENTION

Amyloid β peptides (Aβ) play an important role in the neurodegeneration of Alzheimer's disease (AD). Mutations in the amyloid precursor protein (APP) and the presenilin genes are associated with an increased production of a 42-amino-acid form of Aβ, named Aβ₁₋₄₂, and are linked with exacerbated familial forms of AD (Selkoe 1999 Nature 399 (Suppl):A23). While a direct neurotoxic effect of Aβ has been proposed (Lambert et al. 1998 PNAS USA 95:6448; Yan et al. 1997 PNAS USA 94:5296), the bulk of evidence favors an “indirect” pathway, whereby Aβ induces an inflammatory response in microglia, the brain counterpart of the mononuclear phagocytes (Pachter 1997 Mol Psychiatry 2:91; McGeer & McGeer 1999 J Leukoc Biol 65:400; Kalaria 1999 Curr Opin Hematol 6:15).

Aβ peptides have previously been shown to elicit a diverse array of proinflammatory responses in mononuclear phagocytes. These include induction of cell adhesion, migration (Pachter 1997 Mol Psychiatry 2:91; McGeer & McGeer 1999 J Leukoc Biol 65:400; Kalaria, 1999 Curr Opin Hematol 6:15; Davis et al. 1992 Biochem Biophys Res Commun 189:1096; Klegeris & McGeer 1997 Neurosci Res 49:229; Meda et al. 1996 J Neuroimmunol 93:45; London et al. 1996 PNAS USA 93:4147), accumulation at sites of injection in the brain (Scali et al. 1999 Brain Res 831:319), Ca²⁺ mobilization (Combs et al. 1999 J Neurosci 19:928), phagocytosis (Kopec & Carroll 1998 J Neurochem 71:2123), release of reactive oxygen intermediates, and increased production of neurotoxic or proinflammatory cytokines (Pachter 1997 Mol Psychiatry 2:91; McGeer & McGeer 1999 J Leukoc Biol 65:400; Kalaria 1999 Curr Opin Hematol 6:15; Davis et al. 1992 Biochem Biophys Res Commun 189:1096; Klegeris & McGeer 1997 Neurosci Res 49:229; Meda et al. 1996 J Neuroimmunol 93:45; London et al. 1996 PNAS USA 93:4147; Bonaiuto et al. 1997 J Neuroimmunol 77:51). Aβ signal transduction in monocytes involves activation of G-proteins, protein kinase C (Lorton 1997 Mech Ageing Dev 94:199; Zhang et al. 1996 FEBS Lett 386:185; Nakai et al. 1998 Neuroreport 9:3467; Klegeris et al. 1997 Brain Res 747:114) and tyrosine kinases (McDonald et al. 1997 J Neurosci 17:2284; Liu et al. 1997 Biochem Biophys Res Commun 237:37; McDonald et al. 1998 Biochem Biophys Res Commun 18:4451; Huang et al. 1999 Am J Pathol 155:1741; Combs et al. 1999 J Neurosci 19:928), which are known to be activated by G protein coupled seven transmembrane (STM) receptors (Prossnitz & Ye, 1997 Pharmacol Ther 74:73; Murphy 1994 Annu Rev Immunol 12:593; Ben-Baruch et al. 1995 J Biol Chem 270:11703; Wang et al. 1998 Int J Clin Lab Res 28:83).

According to the “indirect pathway” hypothesis, activated microglia accumulate in and around the senile plaques associated with AD, migrate to these sites, and release neurotoxic mediators in response to Aβ (Davis et al. 1992 Biochem Biophys Res Commun 189:1096; Klegeris & McGeer 1997 Neurosci Res 49:229; Meda et al. 1996 J Neuroimmunol 93:45; London et al. 1996 PNAS. USA 93:4147). Consistent with this hypothesis, subjects receiving anti-inflammatory drugs have shown a significantly delayed onset of AD dementia (Pachter 1997 Mol Psychiatry 2:91; McGeer & McGeer 1999 J Leukoc Biol 65:400; Kalaria 1999 Curr Opin Hematol 6:15).

The search for cellular receptor(s) for Aβ that mediate an inflammatory response is of considerable interest. The scavenger receptor (SR) and the receptor for activated glycation end products (RAGE) (Yan et al. 1996 Nature 382:685; El Khoury et al. 1996 Nature 382:716) bind Aβ, however, it is controversial whether these receptors mediate microglial cell responses (McDonald et al. 1997 J Neurosci 17:2284; Liu et al. 1997 Biochem Biophys Res Commun 237:37; McDonald et al. 1998 Biochem Biophys Res Commun 18:4451; Huang et al. 1999 Am J Pathol 155:1741). Current interest is focused on the identification of a receptor(s) that interacts with Aβ and, in turn, mediates an inflammatory response remains a largely unrealized goal.

### SUMMARY OF THE INVENTION

Amyloid beta (Aβ) is a key contributor to the pathogenesis of Alzheimer's disease (AD). Although Aβ has been reported to be directly neurotoxic, it also causes indirect neuronal damage by activating mononuclear phagocytes (microglia) that accumulate in and around senile plaques. We show that Aβ is a chemotactic agonist for the seven-transmembrane (STM), G-protein coupled receptor named FPRL1 (N-formylpeptide receptor-like 1 receptor), which is expressed on human mononuclear phagocytes. Moreover, FPRL1 is expressed at high levels by inflammatory cells infiltrating senile plaques in brain tissues from AD patients. Thus, FPRL1 mediates inflammation seen in AD and is a target for developing therapeutic agents.

### BRIEF DESCRIPTION OF THE DRAWINGS

FIG. 1 shows activation of human monocytes by Aβ₄₂. A, Migration of monocytes induced by freshly prepared Aβ₄₂ (black bars), Aβ₄₂ “aged” at 37° C. for 3 (diagonally wide hatched bars) or 7 (cross-hatched bars) d, and a peptide with reversed sequence of Aβ₄₂ (diagonally hatched bars, Aβ₄₂₋₁,). *p<0.05 compared with cell migration in the absence of Aβ₄₂. B, Effect of preincubation with medium (black bars) or pertussis toxin (PT) (diagonally hatched bars) (100 ng/ml, 37° C., 30 min) on monocyte migration to fMLF (fMet-Leu-Phe) (100 nM) or Aβ₄₂. *p<0.05 compared with migration of cells cultured in the absence of PT. C, Aβ₄₂-induced Ca²⁺ mobilization in monocytes. Inset, Response of cells treated with PT to 20 μM Aβ₄₂. D, E, Attenuation of Aβ₄₂-induced Ca²⁺ flux by fMLF.

FIG. 2 shows activation of cells expressing FPRL1 (N-formylpeptide receptor-like 1 receptor) and FPR(N-formylpeptide receptor) by Aβ₄₂. A, Ca²⁺ mobilization in FPRL1/293 cells induced by Aβ₄₂ and attenuation by fMLF. B, Signaling of Aβ₄₂ in FPR expressing ETFR (epitope-tagged FPR) cells and attenuation by fMLF. C, Signaling of F peptide (F pep) in FPRL1/293 cells and cross-desensitization with Aβ₄₂.

FIG. 3 shows cell migration induced by Aβ₄₂ via FPR and FPRL1. Dose-dependent migration of FPRL1/293 (black bars) and ETFR cells (diagonally hatched bars) toward Aβ₄₂. fMLF at 100 nM was used as a control. *p<0.05 compared with cell migration to medium.

FIG. 4 shows that amyloid-β is a specific agonist for the G protein-coupled receptor FPR2 (mouse) and its human counterpart FPRL1R (FPR-like 1 receptor). Cells transfected with DNA encoding the receptor indicated at the upper left of each tracing were loaded with Fura-2 and monitored for changes in cell fluorescence, as a reporter of [Ca²⁺]ᵢ, in response to 10 μM Aβ. Control agonists were 10 μM ATP (activating an endogenous nucleotide receptor), 10 μM fMLF (for FPRL1R and FPR2), 5 nM fMLF (for human FPR), 10 nM RANTES (regulated upon activation normal T-cell expressed and secreted), 100 nM fractalkine, and 50 nM 1-309. Test substances were added at the times indicated by the arrowheads. FPR2+PTX (pertussis toxin), FPR2-transfected HEK 293 cells treated with 250 ng/ml pertussis toxin for 4 h before loading with Fura-2. HEK 293, denotes untransfected HEK 293 cells. All results are for HEK 293 cell lines except for CCR8, which is expressed in the mouse pre-B cell lymphoma cell line 4DE4. With the exception of FPR2 and FPRL1R, Aβ given first did not affect the cell response to the control agonist given second. Cell lines were tested at least three times with the exception of Fpr-rs3, which was tested twice, and Fpr-rs1, CCR8, and CX3CR1, which were all tested once. Complete inhibition of Aβ signaling by pertussis toxin was replicated in three independent experiments implying coupling to Gᵢ.

FIG. 5 shows that amyloid-β is a specific agonist for the G protein-coupled receptor FPR2 indicated by desensitization of signaling by other FPR2 agonists. HEK 293 cells expressing FPR2 were loaded with Fura-2 and then monitored for fluorescence changes in sequential stimulation experiments. The name, concentration, and time of the addition of each stimulus are indicated at the arrowheads. The results shown are representative of two independent experiments (A and B) that gave the same pattern.

FIG. 6 shows that amyloid-β is an equipotent calcium-mobilizing agonist at FPR2, FPRL1R, and mouse neutrophils. Agonist activity was measured as the peak of the change in relative fluorescence of Fura-2-loaded HEK 293 cells (A), transfected with the indicated receptors, or neutrophils (B) from FPR+/+ or −/− mice in response to increasing concentrations of Aβ. The results shown in A are representative of three independent experiments. Results in B are the mean±S.E. of three independent experiments, in which each experiment included FPR+/+ and −/− neutrophils and all six concentrations of Aβ.

FIG. 7 shows shared receptor usage by amyloid-β and fMLF in primary mouse neutrophils indicated by cross-desensitization of calcium flux signaling and pertussis toxin sensitivity. Neutrophils from FPR −/− and FPR2+/+mice were loaded with Fura-2 and then monitored for fluorescence changes in sequential stimulation experiments. The name, concentration, and time of the addition of each stimulus are indicated at the arrowheads. Data in A and B are representative of two separate experiments. In C (bottom tracing), cells were incubated in PTX (250 ng/ml) for 4 h and then washed prior to stimulation with Aβ. Data in C are from a single experiment.

FIG. 8 shows that amyloid-β is a potent chemotactic agonist at FPR2. A, chemotaxis versus chemokinesis. FPR2-expressing HEK 293 cells were applied to the upper well of a chemotaxis chamber with or without 10 μM Aβ present in medium. Lower wells contained medium with or without 10 μM Aβ to distinguish chemotaxis from chemokinesis. The contents of the upper and lower wells of the chemotaxis chamber correspond to the numerator and denominator, respectively, of the fraction below each bar in the graph. B, chemotactic potency of Aβ at mouse FPR-transfected (open bars) and FPR2-transfected (solid bars) HEK 293 cells. C, chemotactic potency of Aβ for neutrophils from FPR+/+ (open bars) and −/− (solid bars) mice. All conditions were tested in triplicate, and the results are presented as mean±S.E. Data in A and B are representative of three separate experiments; data in C are representative of two separate experiments.

FIG. 9 shows induction of superoxide generation by amyloid β in mouse neutrophils: FPR independence and desensitization by FPR2-selective concentrations of fMLF. Neutrophils from FPR −/− (A-C) and +/+ (A) mice were stimulated as indicated, and the superoxide produced in the 10 min after the addition of the final substance was measured. Each condition was tested in triplicate, and the results are presented as mean±S.E. A, FPR independence. A representative experiment of two independent experiments is shown. The difference in activity between FPR+/+ and −/− cells was not statistically significant. B and C, desensitization. Cells were stimulated sequentially with 5 μM fMLF and 10 μM A in the order shown. Me₂SO and water are the vehicles for fMLF and Aβ, respectively. The differences in B and C were statistically significant (p<0.05). Representative results of three (B) and two (C) independent experiments are shown.

FIG. 10 shows FPR2 gene expression in mouse brain. Detection of FPR2 mRNA in whole mouse brain by RT-PCR using gene-specific primers is shown. Results are representative of three separate experiments.

FIG. 11 shows that FPR2 mRNA expression and a shared amyloid β/fMLF signaling pathway are induced by lipopolysaccharide (LPS) in the mouse microglial cell line N9. A, FPR2 RNA expression. N9 cells were treated with LPS for the number of hours indicated at the top. mRNA was then isolated and amplified by RT-PCR using FPR2- and β-actin-specific primers as described below. The reaction product for each time point was diluted as shown and visualized by gel electrophoresis. The size of FPR2 and β-actin PCR products is indicated by the arrowheads at the left. B, induction of calcium flux by amyloid-β (Aβ₄₂) in LPS-activated N9 cells. Resting (LPS(−)) and LPS-activated (LPS(+)) N9 cells were loaded with Fura-2 and stimulated with the indicated concentration of Aβ₄₂ at the times indicated by the arrowheads. The results are shown as relative fluorescence in real time. C, induction of chemotaxis by amyloid-β (Aβ₄₂) in LPS-activated N9 cells. The results are expressed as the mean±S.D. chemotactic index (CI), which represents the -fold increase in the number of migrated cells in response to chemoattractants over the spontaneous cell migration (to control medium). Data were obtained by counting the number of migrated cells in three high power fields in triplicate samples. The asterisks indicate p<0.01 for LPS(+) versus LPS(−) at each concentration. D, reciprocal cross-desensitization of calcium flux induction by amyloid-β (Aβ₄₂) and fMLF in LPS-activated N9 cells. Data are real time fluorescence tracings of LPS-activated N9 cells loaded with Fura-2 and stimulated at the times indicated by the arrowheads with the indicated concentrations of agonists. E, Aβ coupling to Gᵢ in N9 microglial cells. LPS-stimulated N9 cells were preincubated in PTX (100 ng/ml) or medium alone at 37° C. for 30 min, washed, and then stimulated with the indicated concentrations of Aβ. The asterisk indicates p<0.01 for the difference between PTX (+) versus PTX (−). Results are representative of three independent experiments.

FIG. 12 shows that Aβ₄₂ induced apoptosis of FPRL1/293 cells and macrophages. Macrophages, FPRL1/293 cells, and parental HEK293 cells were cultured for 48 h with medium alone, 10 μM Aβ₄₂, or 1 μM W pep. After simultaneous staining with annexin-V-FITC and PI, cells were analyzed by flow cytometry. The upper right quadrant represents necrotic cells; the lower right quadrant represents apoptotic cells; the lower left quadrant represents viable, nonapoptotic cells. Numbers denote the cell percentage.

### DETAILED DESCRIPTION OF THE PREFERRED EMBODIMENT

The identification of N-formylpeptide receptor (FPR) class receptor as a functional receptor for amyloid β (Aβ) and detection of FPR class receptor mRNA in mononuclear phagocytes infiltrating senile plaques provide a molecular basis for inflammation in AD. FPR class receptors may mediate the migration and accumulation of mononuclear phagocytes to sites containing elevated Aβ. The infiltrating mononuclear phagocytes may participate in the cell uptake of Aβ through internalization of the ligand-receptor complex. However, the resultant stimulation of the cells by Aβ can promote an inflammatory response characterized by the production of mediators and cytokines that are potentially cytotoxic to neuronal cells. The present invention advances current knowledge about AD pathogenesis and provides an additional target for the development of therapeutic agents.

To date, two FPR class receptors have been identified and cloned (reviewed in: Prossnitz E. R. & R. D. Ye 1997 Pharmacol Ther 74:73-102; and Murphy P. M. 1996 Chemoattractant ligands and their receptors, pp. 269). The prototype FPR class receptor, FPR, binds fMLF (fMet-Leu-Phe) with high affinity and is activated by low concentrations of fMLF. Another highly homologous variant of FPR, named FPRL1 (also referred to as FPRH2 and LXA4R, lipoxin A4 receptor), was originally cloned as an orphan receptor (Murphy et al. 1992 J Biol Chem 267:7637-7643; Ye et al. 1992 Biochem Biophys Res Commun 184:582-589; Bao et al. 1992 Genomics 13:437-440; Gao J. L. & P. M. Murphy 1993 J Biol Chem 268:25395-25401; and Nomura et al. 1993 Int Immunol 5:1239-1249) but was later found to mediate Ca²⁺ mobilization in response to high concentrations of fMLF (Ye et al. 1992 Biochem Biophys Res Commun 184:582-589; and Gao J. L. & P. M. Murphy 1993 J Biol Chem 268:25395-25401). Many more members of the family of FPR class receptors can exist in organisms and these molecules can be identified according to their structure and function. Thus, the term “FPR class receptor” refers to a class of cell surface receptors characterized by their structure, a seven transmembrane (STM), G-protein coupled receptor, and their function, activation of an inflammatory response by ligands such as the bacterial chemotactic peptide N-formyl-methionyl-leucyl-phenylalanine (“fMLP” or “fMLF”), synthetic peptide domains derived from HIV-1 envelope proteins, and endogenously produced ligands, the eicosanoid lipoid A4 (LXA4), serum amyloid A (SAA) and Aβ.

The term “amyloid β” or “Aβ” refers to a bioactive peptide fragment of amyloid β protein. The length of the Aβ fragment can vary and can be indicated by a range of numbers in subscript (e.g., Aβ₁₋₄₃ refers to the 1-43 fragment of the Aβ protein). The sequence of Aβ₁₋₄₃ is provided in the Sequence Listing (SEQ. ID. NO: 1). Several different Aβ fragments are known in the art and can be used with embodiments of the invention. For example, Aβ₁₋₁₆, Aβ₁₋₂₈, Aβ₁₋₃₈, Aβ₁₋₄₀, Aβ₁₋₄₂, Aβ₁₋₄₃, Aβ₁₀₋₂₀, Aβ₁₂₋₂₈, Aβ₂₂₋₃₅, Aβ₂₅₋₃₅, Aβ₃₁₋₃₅, and Aβ₃₂₋₃₅ are commercially available Aβ fragments (SIGMA). Further, the inactive control Aβ peptides which fail to polymerize fibrils Aβ₃₅₋₂₅ and Aβ₄₀₋₁ are available from SIGMA. The inactive Aβ peptides are often used as control reagents. Additionally, an acetyl Aβ fragment 15-20 amide (Aβ_{ac15-20NH2}), which inhibits polymerization of Aβ₁₋₄₀ is commercially available (SIGMA A6933). The sequence of these Aβ fragments can be determined by reference to SEQ. ID. NO: 1. Depending on the context, the term “Aβ” can refer to any or all of these fragments, peptidomimetics that resemble these fragments, or derivatives thereof, and any other fragments, peptidomimetics that resemble Aβ or derivatives thereof that can be constructed by one of skill in the art. These molecules can also be referred to as “binding partners”, a term used to describe molecules that bind to either an FPR class receptor or Aβ.

### Identification of a G-Protein Coupled Receptor FRPL1 as a Link Between Amyloid, and Inflammation in Alzheimer's Disease

We initiated studies by using human peripheral blood monocytes. Freshly dissolved Aβ₄₂ induced dose-dependent migration of human monocytes starting at a concentration of 20 nM. In contrast, the peptide with the reverse sequence of Aβ₄₂ (Aβ₄₂₋₁) was inactive. Checkerboard analysis indicated that Aβ₄₂ functioned chemotactically rather than by increasing random cell migration. Since aggregated Aβ is likely to deposit in senile plaques of AD and activates mononuclear phagocytes in vitro, we tested the chemotactic activity of Aβ₄₂ “aged” at 37° C. This form of Aβ₄₂ also induced significant monocytes migration although with less potency than freshly dissolved peptide. The activation of monocytes by Aβ₄₂ was further demonstrated by an increased Ca²⁺ mobilization. Preincubation of monocytes with pertussis toxin (PT), an inhibitor of Gᵢ-type proteins, completely abolished monocyte migration and calcium flux in response to Aβ₄₂. These results evidence that Aβ₄₂ uses G protein-coupled STM receptor(s) on monocytes.

To identify the monocyte receptor(s) for Aβ₄₂, we examined the capacity of Aβ₄₂ to cross-desensitize signaling with chemoattractants known to elicit Ca²⁺ mobilization. This approach can distinguish between unique and/or shared STM receptors for different chemoattractants. Aβ₄₂ signaling in monocytes was not affected by prior stimulation of the cells with a number of chemokines, suggesting that Aβ₄₂ did not share a chemokine receptor. However, a classical chemoattractant, the bacterial chemotactic peptide formyl-methionyl-leucyl-phenylalanine (fMLF), clearly inhibited the subsequent Ca²⁺ flux response to Aβ₄₂ in a concentration-dependent manner. Since high concentrations of fMLF were required, we postulated that Aβ₄₂ might share a low affinity fMLF receptor. Such a receptor was cloned ten years ago and has been designated FPRL1 or LXA4R based on its homology to the high affinity fMLF receptor FPR, and its reported function as a lipoxin A4 receptor. Moreover, FPRL1 in a previous study has been identified as a functional receptor for serum amyloid A (SAA) which is chemotactic for human leukocytes and is one of the major amyloidogenic proteins during chronic inflammation in various organs and tissues, but has not been implicated in AD.

We therefore tested the capacity of Aβ₄₂ to activate cells transfected to express solely FPRL1 or FPR. Aβ₄₂ dose-dependently induced Ca²⁺ mobilization in FPRL1 transfected human embryonic kidney 293 cell (FPRL1/293 cells). Aβ₄₂ also induced Ca²⁺ mobilization in a rat basophilic leukemia cell line transfected with FPR (ETFR cells), yet with much lower potency and efficacy than fMLF. Aβ₄₂ signaling was dependent on FPRL1 and FPR, since untransfected parental cells or cells transfected with other chemoattractant receptors did not respond. Consistent with the monocyte experiments, Aβ₄₂ signaling in both FPRL1/293 and ETFR cells was desensitized by prior-stimulation of the cells with fMLF. In addition, a derivative of the HIV-1 envelope protein domain named F peptide, which specifically activates FPRL1, desensitized Aβ₄₂-induced Ca²⁺ flux in FPRL1/293 cells and vice versa. Furthermore, FPRL1/293 cells exhibited a potent chemotactic response to Aβ₄₂, whereas ETFR cells migrated only weakly, albeit significantly, in response to high concentrations of Aβ₄₂. Since directional cell migration is considered as an initial step for cell infiltration and accumulation at sites of inflammation, our results evidence that FPRL1 is a physiologically relevant receptor used by Aβ₄₂.

To gain insight into the pathophysiological relevance of FPRL1 to AD, we examined FPRL1 gene expression in normal versus AD brain tissues. Multiple senile plaques were readily visible on sections of brain tissues from AD patients, but not from normal brain. All senile plaques, but not surrounding brain tissue, were infiltrated by cells expressing considerable level of FPRL1 as determined by in situ hybridization with antisense mRNA transcribed from the FPRL1 cDNA. In contrast, hybridization signals were not detected with FPRL1 sense mRNA in serial sections of senile plaques. By immunohistochemistry, we also detected a large number of infiltrating cells that were positively stained with monoclonal antibody against CD11b, a marker for mononuclear phagocytes at the sites of AD lesions.

We additionally tested the effect of ligands for SR or receptor for advanced glycation end products (RAGE) on monocytes and receptor transfected cells. Glycated bovine albumin (GlyBSA), a reported ligand for RAGE, was a potent inducer of Ca²⁺ mobilization in human monocytes, and was able to cross-desensitize the cell response to Aβ₄₂ and vice versa. In contrast, fucoidan, a SR ligand, did not induce Ca²⁺ mobilization in monocytes, and did not affect monocyte response to subsequent stimulation with Aβ₄₂. Since the signaling of GlyBSA in monocytes was also desensitized by fMLF, we examined the capacity of GlyBSA to directly activate fMLF receptors. Similar to fMLF, GlyBSA stimulated Ca²⁺ mobilization both in FPRL1 and FPR transfected cells but not in parental cells or cells transfected with chemokine receptors. The signaling of GlyBSA in FPRL1/293 cells was desensitized by Aβ₄₂ and vice versa. GlyBSA and fMLF also cross-desensitized one another in both FPRL1/293 and ETFR cells. These results evidence that Aβ₄₂-induced Ca²⁺ flux in monocytes is independent of the SR or RAGE and furthermore, that the reported RAGE ligand GlyBSA is an agonist for both FPRL1 and FPR.

The invention encompasses the use of FPR class receptor nucleotides, FPR class receptor proteins and peptides, as well as antibodies to the FPR class receptor (which can, for example, act as FPR class receptor agonists or antagonists), antagonists that inhibit receptor activity or expression, or agonists that activate receptor activity or increase its expression in the diagnosis and treatment of inflammation, including inflammation in Alzheimer's disease (AD), in humans. The diagnosis of an FPR class receptor abnormality in a patient, or an abnormality in the FPR class receptor signal transduction pathway, will assist in devising a proper treatment or therapeutic regimen. In addition, FPR class receptor nucleotides and FPR class receptor proteins are useful for the identification of compounds effective in the treatment of inflammation, including inflammation in AD.

In particular, the invention encompasses FPR class receptor, polypeptides or peptides corresponding to functional domains of the FPR class receptor, mutated, truncated or deleted FPR class receptors, FPR class receptor fusion proteins, nucleotide sequences encoding such products, and host cell expression systems that can produce such FPR class receptor products.

The invention also encompasses antibodies and anti-idiotypic antibodies (including Fab fragments), antagonists and agonists of the FPR class receptor, as well as compounds or nucleotide constructs that inhibit expression of the FPR class receptor gene (transcription factor inhibitors, antisense and ribozyme molecules, or gene or regulatory sequence replacement constructs), or promote expression of FPR class receptor (e.g., expression constructs in which FPR class receptor coding sequences are operatively associated with expression control elements such as promoters, promoter/enhancers, etc.). The invention also relates to host cells and animals genetically engineered to express the human FPR class receptor (or mutants thereof) or to inhibit or “knock-out” expression of the animal's endogenous FPR class receptor.

The FPR class receptor proteins or peptides, FPR class receptor fusion proteins, FPR class receptor nucleotide sequences, antibodies, antagonists and agonists can be useful for the detection of mutant FPR class receptors or inappropriately expressed FPR class receptors for the diagnosis of inflammation, including inflammation in AD. The FPR class receptor proteins or peptides, FPR class receptor fusion proteins, FPR class receptor nucleotide sequences, host cell expression systems, antibodies, antagonists, agonists and genetically engineered cells and animals can be used for screening for drugs effective in the treatment of such inflammatory disorders. The use of engineered host cells and/or animals may offer an advantage in that such systems allow not only for the identification of compounds that bind to the FPR class receptor, but can also identify compounds that affect the signal transduced by the activated FPR class receptor.

Finally, the FPR class receptor protein products (especially soluble derivatives) and fusion protein products, antibodies and anti-idiotypic antibodies (including Fab fragments), antagonists or agonists (including compounds that modulate signal transduction which may act on downstream targets in the FPR class receptor signal transduction pathway) can be used for therapy of such diseases. For example, the administration of an effective amount of soluble FPR class receptor or a fusion protein or an anti-idiotypic antibody (or its Fab) that mimics the FPR class receptor would “mop up” or “neutralize” endogenous Aβ, and prevent or reduce binding and receptor activation, leading to inflammation, including inflammation in AD. Nucleotide constructs encoding such FPR class receptor products can be used to genetically engineer host cells to express such FPR class receptor products in vivo; these genetically engineered cells function as “bioreactors” in the body delivering a continuous supply of the FPR class receptor, FPR class receptor peptide, soluble or FPR class receptor fusion protein that will “mop up” or neutralize Aβ. Nucleotide constructs encoding functional FPR class receptors, mutant FPR class receptors, as well as antisense and ribozyme molecules can be used in “gene therapy” approaches for the modulation of FPR class receptor expression and/or activity in the treatment of inflammation, including inflammation in AD. Thus, the invention also encompasses pharmaceutical formulations and methods for treating inflammation, including inflammation in AD.

The term “isolated” requires that a material be removed from its original environment (e.g., the natural environment if it is naturally occurring). For example, a naturally occurring polynucleotide or polypeptide present in a living cell is not isolated, but the same polynucleotide or polypeptide, separated from some or all of the coexisting materials in the natural system, is isolated. The term “purified” does not require absolute purity; rather it is intended as a relative definition, with reference to the purity of the material in its natural state. Purification of natural material to at least one order of magnitude, preferably two or three orders of magnitude, and more preferably four or five orders of magnitude is expressly contemplated. The term “enriched” means that the concentration of the material is at least about 2, 5, 10, 100, or 1000 times its natural concentration (for example), advantageously 0.01% by weight. Enriched preparations of about 0.5%, 1%, 5%, 10%, and 20% by weight are also contemplated.

Various aspects of the invention are described in greater detail in the subsections below.

### The FPR Class Receptor Gene

The gene sequence, cDNA sequence and deduced amino acid sequence of FPR class receptors are known (Boulay et al. 1990 Biochemistry 29:11123; Murphy et al. 1992 J Biol Chem 267:7637; Ye et al. 1992 Biochem Biophys Res Commun 184:582; De Nardin et al. 1992 Biochem Int 26:381; Perez et al. 1992 Biochemistry 31:11595; Bao et al. 1992 Genomics 13:437; Murphy et al. 1993 Gene 133:285; Takano et al. 1997 J Exp Med 185:1693; Gao et al. 1998 Genomics 51:270).

The FPR class receptor nucleotide sequences of the invention include: (a) the gene sequence; (b) the cDNA sequence; (c) the nucleotide sequence that encodes the amino acid sequence (d) any nucleotide sequence that hybridizes to the complement of the DNA sequence encoding an FPR class receptor under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al. eds. 1989 Current Protocols in Molecular Biology Vol. I, Green Publishing Associates, Inc., and John Wiley & sons, Inc., New York, at p. 2.10.3) and encodes a functionally equivalent gene product; and (e) any nucleotide sequence that hybridizes to the complement of the DNA sequences that encode the amino acid sequence of an FPR class receptor under moderately stringent conditions, e.g., washing in 0.2×SSC/0.1% SDS at 42° C. (Ausubel et al. eds. 1989 Current Protocols in Molecular Biology Vol. I, Green Publishing Associates, Inc., and John Wiley & sons, Inc., New York, at p. 2.10.3), yet which still encodes a functionally equivalent FPR class receptor gene product. Functional equivalents of the FPR class receptor include naturally occurring FPR present in other species, and mutant FPR class receptors whether naturally occurring or engineered. The invention also includes degenerate variants of sequences (a) through (e).

The invention also includes nucleic acid molecules, preferably DNA molecules, that hybridize to, and are therefore the complements of, the nucleotide sequences (a) through (e), in the preceding paragraph. Such hybridization conditions may be highly stringent or moderately stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as FPR class receptor antisense molecules, useful, for example, in FPR class receptor gene regulation (for and/or as antisense primers in amplification reactions of FPR class receptor gene nucleic acid sequences). With respect to FPR class receptor gene regulation, such techniques can be used to regulate, for example, inflammation, including inflammation in AD. Further, such sequences may be used as part of ribozyme and/or triple helix sequences, also useful for FPR class receptor gene regulation. Still further, such molecules may be used as components of diagnostic methods whereby, for example, the presence of a particular FPR class receptor allele responsible for causing inflammation, including inflammation in AD, may be detected.

In addition to the FPR class receptor nucleotide sequences described above, full length FPR class receptor cDNA or gene sequences present in the same species and/or homologs of the FPR class receptor gene present in other species can be identified and readily isolated, without undue experimentation, by molecular biological techniques well known in the art. The identification of homologs of FPR in related species can be useful for developing animal model systems more closely related to humans for purposes of drug discovery. For example, expression libraries of cDNAs synthesized from brain tissue mRNA derived from the organism of interest can be screened using labeled Aβ derived from that species, e.g., an Aβ fusion protein. Alternatively, such cDNA libraries, or genomic DNA libraries derived from the organism of interest can be screened by hybridization using the nucleotides described herein as hybridization or amplification probes. Furthermore, genes at other genetic loci within the genome that encode proteins which have extensive homology to one or more domains of the FPR class receptor gene product can also be identified via similar techniques. In the case of cDNA libraries, such screening techniques can identify clones derived from alternatively spliced transcripts in the same or different species.

Screening can be by filter hybridization, using duplicate filters. The labeled probe can contain at least 15-30 base pairs of the FPR class receptor nucleotide sequence. The hybridization washing conditions used should be of a lower stringency when the cDNA library is derived from an organism different from the type of organism from which the labeled sequence was derived. With respect to the cloning of a human FPR class receptor homolog, using murine FPR probes, for example, hybridization can, for example, be performed at 65° C. overnight in Church's buffer (7% SDS, 250 mM NaHPO₄, 2 μM EDTA, 1% BSA). Washes can be done with 2×SSC, 0.1% SDS at 65° C. and then at 0.1×SSC, 0.1% SDS at 65° C.

Low stringency conditions are well known to those of skill in the art, and will vary predictably depending on the specific organisms from which the library and the labeled sequences are derived. For guidance regarding such conditions see, for example, Sambrook et al. 1989 Molecular Cloning, A Laboratory Manual, Cold Springs Harbor Press, N.Y.; and Ausubel et al. 1989 Current Protocols in Molecular Biology, Green Publishing Associates and Wiley Interscience, N.Y.

Alternatively, the labeled FPR class receptor nucleotide probe may be used to screen a genomic library derived from the organism of interest, again, using appropriately stringent conditions. The identification and characterization of human genomic clones is helpful for designing diagnostic tests and clinical protocols for treating inflammation, including inflammation in AD, in human patients. For example, sequences derived from regions adjacent to the intron/exon boundaries of the human gene can be used to design primers for use in amplification assays to detect mutations within the exons, introns, splice sites (e.g., splice acceptor and/or donor sites)etc., that can be used in diagnostics.

Further, an FPR class receptor gene homolog may be isolated from nucleic acid of the organism of interest by performing PCR using two degenerate oligonucleotide primer pools designed on the basis of amino acid sequences within the FPR class receptor gene product disclosed herein. The template for the reaction may be cDNA obtained by reverse transcription of mRNA prepared from, for example, human or non-human cell lines or tissue, such as brain tissue, known or suspected to express an FPR class receptor gene allele.

The PCR product may be subcloned and sequenced to ensure that the amplified sequences represent the sequences of an FPR class receptor gene. The PCR fragment may then be used to isolate a full-length cDNA clone by a variety of methods. For example, the amplified fragment may be labeled and used to screen a cDNA library, such as a bacteriophage cDNA library. Alternatively, the labeled fragment may be used to isolate genomic clones via the screening of a genomic library.

PCR technology may also be utilized to isolate full-length cDNA sequences. For example, RNA may be isolated, following standard procedures, from an appropriate cellular or tissue source (i.e., one known, or suspected, to express the FPR class receptor gene, such as, for example, brain tissue). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al. 1989 Molecular Cloning, A Laboratory Manual, Cold Springs Harbor Press, N.Y.

The FPR class receptor gene sequences may additionally be used to isolate mutant FPR class receptor gene alleles. Such mutant alleles may be isolated from individuals either known or proposed to have a genotype which contributes to the symptoms of inflammation, including inflammation in AD. Mutant alleles and mutant allele products may then be utilized in the therapeutic and diagnostic systems described below. Additionally, such FPR class receptor gene sequences can be used to detect FPR class receptor gene regulatory (e.g., promoter or promotor/enhancer) defects which can affect inflammation, including inflammation in AD.

A cDNA of a mutant FPR class receptor gene may be isolated, for example, by using PCR, a technique which is well known to those of skill in the art. In this case, the first cDNA strand may be synthesized by hybridizing an oligo-dT oligonucleotide to mRNA isolated from tissue known or suspected to be expressed in an individual putatively carrying the mutant FPR class receptor allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant FPR class receptor allele to that of the normal FPR class receptor allele, the mutation(s) responsible for the loss or alteration of function of the mutant FPR class receptor gene product can be ascertained.

Alternatively, a genomic library can be constructed using DNA obtained from an individual suspected of or known to carry the mutant FPR class receptor allele, or a cDNA library can be constructed using RNA from a tissue known, or suspected, to express the mutant FPR class receptor allele. The normal FPR class receptor gene or any suitable fragment thereof may then be labeled and used as a probe to identify the corresponding mutant FPR class receptor allele in such libraries. Clones containing the mutant FPR class receptor gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant FPR class receptor allele in an individual suspected of or known to carry such a mutant allele. In this manner, gene products made by the putatively mutant tissue may be expressed and screened using standard antibody screening techniques in conjunction with antibodies raised against the normal FPR class receptor gene product, as described, below. (For screening techniques, see, for example, Harlow E. & Lane eds. 1988 Antibodies: A Laboratory Manual Cold Spring Harbor Press, Cold Spring Harbor.) Additionally, screening can be accomplished by screening with labeled Aβ fusion proteins. In cases where an FPR class receptor mutation results in an expressed gene product with altered function (e.g., as a result of a missense or a frameshift mutation), a polyclonal set of antibodies to FPR class receptor are likely to cross-react with the mutant FPR class receptor gene product. Library clones detected via their reaction with such labeled antibodies can be purified and subjected to sequence analysis according to methods well known to those of skill in the art.

The invention also encompasses nucleotide sequences that encode mutant FPR class receptors, peptide fragments of the FPR class receptor, truncated FPR class receptors, and FPR class receptor fusion proteins. These include, but are not limited to nucleotide sequences encoding mutant FPR class receptors described in sections below; polypeptides or peptides corresponding to domains of the FPR class receptor or portions of these domains; truncated FPR class receptors in which one or two of the domains is deleted, e.g., a soluble FPR class receptor lacking the transmembrane domain or both the transmembrane domain and the cytoplasmic domain or a truncated, nonfunctional FPR class receptor lacking all or a portion of the cytoplasmic domain. Nucleotides encoding fusion proteins may include but are not limited to full length FPR class receptor, truncated FPR class receptor or peptide fragments of FPR class receptor fused to an unrelated protein or peptide, such as for example, a transmembrane sequence, which anchors the FPR class receptor extra-cellular domain to the cell membrane; a domain which increases the stability and half life of the resulting fusion protein (e.g., FPR class receptor-Ig) in the bloodstream; or an enzyme, fluorescent protein, luminescent protein which can be used as a marker.

The invention also encompasses (a) DNA vectors that contain any of the foregoing FPR class receptor coding sequences and/or their complements (i.e., antisense); (b) DNA expression vectors that contain any of the foregoing FPR class receptor coding sequences operatively associated with a regulatory element that directs the expression of the coding sequences; and (c) genetically engineered host cells that contain any of the foregoing FPR class receptor coding sequences operatively associated with a regulatory element that directs the expression of the coding sequences in the host cell. As used herein, regulatory elements include but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Particular polynucleotides are DNA sequences having three sequential nucleotides, four sequential nucleotides, five sequential nucleotides, six sequential nucleotides, seven sequential nucleotides, eight sequential nucleotides, nine sequential nucleotides, ten sequential nucleotides, eleven sequential nucleotides, twelve sequential nucleotides, thirteen sequential nucleotides, fourteen sequential nucleotides, fifteen sequential nucleotides, sixteen sequential nucleotides, seventeen sequential nucleotides, eighteen sequential nucleotides, nineteen sequential nucleotides, twenty sequential nucleotides, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty,-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven, sixty-eight, sixty-nine, seventy, seventy-one, seventy-two, seventy-three, seventy-four, seventy-five, seventy-six, seventy-seven, seventy-eight, seventy-nine, eighty, ninety, one-hundred, two-hundred, or three-hundred or more sequential nucleotides.

### FPR Class Receptor Proteins and Polypeptides

FPR class receptor protein, polypeptides and peptide fragments, mutated, truncated or deleted forms of the FPR class receptor and/or FPR class receptor fusion proteins can be prepared for a variety of uses, including but not limited to the generation of antibodies, as reagents in diagnostic assays, the identification of other cellular gene products involved in the regulation of inflammation, including inflammation in AD, as reagents in assays for screening for compounds that can be used in the treatment of inflammation, including inflammation in AD, and as pharmaceutical reagents useful in the treatment of inflammation, including inflammation in AD, related to the FPR class receptor.

The FPR class receptor amino acid sequences of the invention include the amino acid sequence, or the amino acid sequence encoded by the cDNA or encoded by the gene. Further, FPR class receptors of other species are encompassed by the invention. In fact, any FPR class receptor protein encoded by the FPR class receptor nucleotide sequences described in the section above is within the scope of the invention.

The invention also encompasses proteins that are functionally equivalent to the FPR class receptor encoded by the nucleotide sequences described in the section above, as judged by any of a number of criteria, including but not limited to the ability to bind Aβ, the binding affinity for Aβ, the resulting biological effect of Aβ binding, e.g., signal transduction, a change in cellular metabolism (e.a., ion flux, tyrosine phosphorylation) or change in phenotype when the FPR class receptor equivalent is present in an appropriate cell type (such as the amelioration, prevention or delay of the AD phenotype), or inflammation, including inflammation in AD. Such functionally equivalent FPR class receptor proteins include but are not limited to additions or substitutions of amino acid residues within the amino acid sequence encoded by the FPR class receptor nucleotide sequences described, above, in the section, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. While random mutations can be made to FPR class receptor DNA (using random mutagenesis techniques well known to those skilled in the art) and the resulting mutant FPR class receptors tested for activity, site-directed mutations of the FPR class receptor coding sequence can be engineered (using site-directed mutagenesis techniques well known to those skilled in the art) to generate mutant FPR class receptors with increased function, e.g., higher binding affinity for Aβ, and/or greater signalling capacity; or decreased function, e.g., lower binding affinity for Aβ, and/or decreased signal transduction capacity.

For example, the identical amino acid residues of FPR and FPRL1 can be aligned. Mutant FPR class receptors can be engineered so that regions of identity are maintained, whereas the variable residues are altered, e.g., by deletion or insertion of an amino acid residue(s) or by substitution of one or more different amino acid residues. Conservative alterations at the variable positions can be engineered in order to produce a mutant FPR class receptor that retains function; e.g., Aβ binding affinity or signal transduction capability or both. Non-conservative changes can be engineered at these variable positions to alter function, e.g., Aβ binding affinity or signal transduction capability, or both. Alternatively, where alteration of function is desired, deletion or non-conservative alterations of the conserved regions (i.e., identical amino acids) can be engineered. For example, deletion or non-conservative alterations (substitutions or insertions) of the cytoplasmic domain or portions of the cytoplasmic domain can be engineered to produce a mutant FPR class receptor that binds AD but is signalling-incompetent. Non-conservative alterations to residues of identical amino acids in the extra-cellular domain can be engineered to produce mutant FPR class receptors with altered binding affinity for Aβ. The same mutation strategy can also be used to design mutant FPR class receptors based on the alignment of non-human FPR class receptor and the human FPR class receptor homolog by aligning identical amino acid residues.

Other mutations to the FPR class receptor coding sequence can be made to generate FPR class receptors that are better suited for expression, scale up, etc. in the host cells chosen. For example, cysteine residues can be deleted or substituted with another amino acid in order to eliminate disulfide bridges; N-linked glycosylation sites can be altered or eliminated to achieve, for example, expression of a homogeneous product that is more easily recovered and purified from yeast hosts which are known to hyperglycosylate N-linked sites (see, e.g., Miyajima et al. 1986 EMBO J 5:1193-1197).

Peptides corresponding to one or more domains of the FPR class receptor (e.g., extra-cellular domain, transmembrane domain, or cytoplasmic domain), truncated or deleted FPR class receptors (e.g., FPR class receptor in which the transmembrane domain and/or cytoplasmic domain is deleted) as well as fusion proteins in which the full length FPR class receptor, an FPR class receptor peptide or truncated FPR class receptor is fused to an unrelated protein are also within the scope of the invention and can be designed on the basis of the FPR class receptor nucleotide and FPR class receptor amino acid sequences. Such fusion proteins include but are not limited to IgFc fusions which stabilize the FPR class receptor protein or peptide and prolong half-life in vivo; or fusions to any amino acid sequence that allows the fusion protein to be anchored to the cell membrane, allowing the extra-cellular domain to be exhibited on the cell surface; or fusions to an enzyme, fluorescent protein, or luminescent protein which provide a marker function.

While the FPR class receptor polypeptides and peptides can be chemically synthesized (e.g., see Creighton 1983 Proteins: Structures and Molecular Principles W. H. Freeman & Co., N.Y.), large polypeptides derived from the FPR class receptor and the full length FPR class receptor itself may advantageously be produced by recombinant DNA technology using techniques well known in the art for expressing nucleic acid containing FPR class receptor gene sequences and/or coding sequences. Such methods can be used to construct expression vectors containing the FPR class receptor nucleotide sequences described in the section above and appropriate transcriptional and translational control signals. These methods include, for example, in vitro recombinant DNA techniques, synthetic techniques, and in vivo genetic recombination. See, for example, the techniques described in Sambrook et al. 1989 Molecular Cloning, A Laboratory Manual, Cold Springs Harbor Press, N.Y.; and Ausubel et al. 1989 Current Protocols in Molecular Biology, Green Publishing Associates and Wiley Interscience, N.Y. Alternatively, RNA capable of encoding FPR class receptor nucleotide sequences may be chemically synthesized using, for example, synthesizers. See, for example, the techniques described in Gait M. J. ed. 1984 Oligonucleotide Synthesis IRL Press, Oxford, which is incorporated by reference herein in its entirety.

A variety of host-expression vector systems may be utilized to express the FPR class receptor nucleotide sequences of the invention. Where the FPR class receptor peptide or polypeptide is a soluble derivative (e.g., FPR class receptor peptides corresponding to the extra-cellular domain; truncated or deleted FPR class receptor in which the transmembrane and/or cytoplasmic domain are deleted) the peptide or polypeptide can be recovered from the culture, i.e., from the host cell in cases where the FPR class receptor peptide or polypeptide is not secreted, and from the culture media in cases where the FPR class receptor peptide or polypeptide is secreted by the cells. However, the expression systems also encompass engineered host cells that express the FPR class receptor or functional equivalents in situ, i.e., anchored in the cell membrane. Purification or enrichment of the FPR class receptor from such expression systems can be accomplished using appropriate detergents and lipid micelles and methods well known to those skilled in the art. However, such engineered host cells themselves may be used in situations where it is important not only to retain the structural and functional characteristics of the FPR class receptor, but to assess biological activity, e.g., in drug screening assays.

The expression systems that may be used for purposes of the invention include but are not limited to microorganisms such as bacteria (e.g., E. coli, B. subtilis) transformed with recombinant bacteriophage DNA, plasmid DNA or cosmid DNA expression vectors containing FPR class receptor nucleotide sequences; yeast (e.g., Saccharomyces, Pichia) transformed with recombinant yeast expression vectors containing the FPR class receptor nucleotide sequences; insect cell systems infected with recombinant virus expression vectors (e.g., baculovirus) containing the FPR class receptor sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing FPR class receptor nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the FPR class receptor gene product being expressed. For example, when a large quantity of such a protein is to be produced, for the generation of pharmaceutical compositions of FPR class receptor protein or for raising antibodies to the FPR class receptor protein, for example, vectors which direct the expression of high levels of fusion protein products that are readily purified may be desirable. Such vectors include, but are not limited, to the E. coli expression vector pUR278 (Ruther et al. 1983 EMBO J. 2:1791), in which the FPR class receptor coding sequence may be ligated individually into the vector in frame with the lacZ coding region so that a fusion protein is produced; pIN vectors (Inouye & Inouye 1985 Nucleic Acids Res 13:3101-3109; Van Heeke & Schuster 1989 J Biol Chem 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. The FPR class receptor gene coding sequence may be cloned individually into non-essential regions (for example the polyhedrin gene) of the virus and placed under control of an AcNPV promoter (for example the polyhedrin promoter). Successful insertion of FPR class receptor gene coding sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed (e.g., see Smith et al. 1983 J Virol 46: 584; Smith, U.S. Pat. No. 4,215,051).

In mammalian host cells, a number of viral-based expression systems may be utilized. In cases where an adenovirus is used as an expression vector, the FPR class receptor nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the FPR class receptor gene product in infected hosts (e.g., see Logan & Shenk 1984 PNAS USA 81:3655-3659). Specific initiation signals may also be required for efficient translation of inserted FPR class receptor nucleotide sequences. These signals include the ATG initiation codon and adjacent sequences. In cases where an entire FPR class receptor gene or cDNA, including its own initiation codon and adjacent sequences, is inserted into the appropriate expression vector, no additional translational control signals may be needed. However, in cases where only a portion of the FPR class receptor coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al. 1987 Methods in Enzymol 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, and in particular, brain tissue cell lines.

For long-term, high-yield production of recombinant proteins, stable expression is preferred. For example, cell lines which stably express the FPR class receptor sequences described above may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the FPR class receptor gene product. Such engineered cell lines may be particularly useful in screening and evaluation of compounds that affect the endogenous activity of the FPR class receptor gene product.

A number of selection systems may be used, including but not limited to the herpes simplex virus thymidine kinase (Wigler et al. 1977 Cell 11:223), hypoxanthine-guanine phosphoribosyltransferase (Szybalska & Szybalski 1962 PNAS USA 48:2026), and adenine phosphoribosyltransferase (Lowy et al. 1980 Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler et al. 1980 PNAS USA 77:3567; O'Hare et al. 1981 PNAS USA 78:1527); gpt, which confers resistance to mycophenolic acid (Mulligan & Berg 1981 PNAS USA 78:2072); neo, which confers resistance to the aminoglycoside G-418 (Colberre-Garapin et al. 1981 J Mol Biol 150:1); and hygro, which confers resistance to hygromycin (Santerre et al. 1984 Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al. 1991 PNAS USA 88:8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The FPR class receptor gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate FPR class receptor transgenic animals.

Any technique known in the art may be used to introduce the FPR class receptor transgene into animals to produce the founder lines of transgenic animals. Such techniques include, but are not limited to pronuclear microinjection (Hoppe P. C. & Wagner T. E. 1989 U.S. Pat. No. 4,873,191); retrovirus mediated gene transfer into germ lines (Van der Putten et al. 1985 PNAS USA 82:6148-6152); gene targeting in embryonic stem cells (Thompson et al. 1989 Cell 56:313-321); electroporation of embryos (Lo 1983 Mol Cell Biol 3:1803-1814); and sperm-mediated gene transfer (Lavitrano et al. 1989 Cell 57:717-723); etc. For a review of such techniques, see Gordon 1989 Intl Rev Cytol 115:171-229, which is incorporated by reference herein in its entirety.

The present invention provides for transgenic animals that carry the FPR class receptor transgene in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko M. et al. 1992 PNAS USA 89:6232-6236). The regulatory sequences required for such a cell type-specific activation will depend upon the particular cell type of interest, and will be apparent to those of skill in the art. When it is desired that the FPR class receptor gene transgene be integrated into the chromosomal site of the endogenous FPR class receptor gene, gene targeting is preferred. Briefly, when such a technique is to be utilized, vectors containing some nucleotide sequences homologous to the endogenous FPR class receptor gene are designed for the purpose of integrating, via homologous recombination with chromosomal sequences, into and disrupting the function of the nucleotide sequence of the endogenous FPR class receptor gene. The transgene may also be selectively introduced into a particular cell type, thus inactivating the endogenous FPR class receptor gene in only that cell type, by following, for example, the teaching of Gu et al. (Gu et al. 1994 Science 265:103-106). The regulatory sequences required for such a cell-type specific inactivation will depend upon the particular cell type of interest, and will be apparent to those of skill in the art.

Once transgenic animals have been generated, the expression of the recombinant FPR class receptor gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of FPR class receptor gene-expressing tissue, may also be evaluated immunocytochemically using antibodies specific for the FPR class receptor transgene product.

Particular polypeptides are amino acid sequences having three sequential residues, four sequential residues, five sequential residues, six sequential residues, seven sequential residues, eight sequential residues, nine sequential residues, ten sequential residues, eleven sequential residues, twelve sequential residues, thirteen sequential residues, fourteen sequential residues, fifteen sequential residues, sixteen sequential residues, seventeen sequential residues, eighteen sequential residues, nineteen sequential residues, twenty sequential residues, twenty-one, twenty-two, twenty-three, twenty-four, twenty-five, twenty-six, twenty-seven, thirty, forty, fifty, sixty, seventy, eighty, ninety, or more sequential residues.

### Antibodies to FPR Class Receptor Proteins

Antibodies that specifically recognize one or more epitopes of FPR class receptor, or epitopes of conserved variants of FPR class receptor, or peptide fragments of the FPR class receptor are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of the FPR class receptor in a biological sample and may, therefore, be utilized as part of a diagnostic or prognostic technique whereby patients may be tested for abnormal amounts of FPR class receptor. Such antibodies may also be utilized in conjunction with, for example, compound screening schemes, as described in the section below for the evaluation of the effect of test compounds on expression and/or activity of the FPR class receptor gene product. Additionally, such antibodies can be used in conjunction with the gene therapy techniques described in the section below to, for example, evaluate the normal and/or engineered FPR class receptor-expressing cells prior to their introduction into the patient. Such antibodies may additionally be used as a method for the inhibition of abnormal FPR class receptor activity. Thus, such antibodies may, therefore, be utilized as part of methods for treatment of inflammation, including inflammation in AD.

For the production of antibodies, various host animals may be immunized by injection with the FPR class receptor, an FPR class receptor peptide (e.g., one corresponding the a functional domain of the receptor, such as extra-cellular domain, transmembrane domain or cytoplasmic domain), truncated FPR class receptor polypeptides (FPR class receptor in which one or more domains, e.g., the transmembrane or cytoplasmic domain, has been deleted), functional equivalents of the FPR class receptor or mutants of the FPR class receptor. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein (1975 Nature 256:495-497; and U.S. Pat. No. 4,376,110), the human B-cell hybridoma technique (Kosbor et al. 1983 Immunology Today 4:72; Cole et al. 1983 PNAS USA 80:2026-2030), and the EBV-hybridoma technique (Cole et al. 1985 Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al. 1984 PNAS USA 81:6851-6855; Neuberger et al. 1984 Nature 312:604-608; Takeda et al. 1985 Nature 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird 1988 Science 242:423-426; Huston et al. 1988 PNAS USA 85:5879-5883; and Ward et al. 1989 Nature 334:544-546) can be adapted to produce single chain antibodies against FPR class receptor gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al. 1989 Science 246:1275-1281) to allow rapid and easy identification of monoclonal Fab fragments with the desired specificity.

Antibodies to the FPR class receptor can, in turn, be utilized to generate anti-idiotype antibodies that “mimic” the FPR class receptor, using techniques well known to those skilled in the art (see, e.g., Greenspan & Bona 1993 FASEB J 7:437-444; and Nissinoff 1991 J Immunol 147:2429-2438). For example antibodies which bind to the FPR class receptor extra-cellular domain and competitively inhibit the binding of Aβ to the FPR class receptor can be used to generate anti-idiotypes that “mimic” the extra-cellular domain and, therefore, bind and neutralize Aβ. Such neutralizing anti-idiotypes or Fab fragments of such anti-idiotypes can be used in therapeutic regimens to neutralize Aβ and inhibit inflammation, including inflammation in AD.

### Diagnosis of Inflammatory Disorder Abnormalities, Including Inflammation in AD

A variety of methods can be employed for the diagnostic and prognostic evaluation of inflammation, including inflammation in AD, and for the identification of subjects having a predisposition to such disorders.

Such methods may, for example, utilize reagents such as the FPR class receptor nucleotide sequences described in the section above, and FPR class receptor antibodies, as described in the section above. Specifically, such reagents may be used, for example, for: (1) the detection of the presence of FPR class receptor gene mutations, or the detection of either over- or under-expression of FPR class receptor mRNA relative to the non-inflammatory disorder state; (2) the detection of either an over- or an under-abundance of FPR class receptor gene product relative to the non-inflammatory disorder state; and (3) the detection of perturbations or abnormalities in the signal transduction pathway mediated by FPR class receptor.

The methods described herein may be performed, for example, by utilizing pre-packaged diagnostic kits comprising at least one specific FPR class receptor nucleotide sequence or FPR class receptor antibody reagent described herein, which may be conveniently used, e.g., in clinical settings, to diagnose patients exhibiting inflammatory disorder abnormalities, including inflammation in AD.

For the detection of FPR class receptor mutations, any nucleated cell can be used as a starting source for genomic nucleic acid. For the detection of FPR class receptor gene expression or FPR class receptor gene products, any cell type or tissue in which the FPR class receptor gene is expressed, such as, for example, brain tissue cells, may be utilized.

Nucleic acid-based detection techniques are described below. Peptide detection techniques are also described below.

### Detection of FPR Class Receptor Gene and Transcripts

Mutations within the FPR class receptor gene can be detected by utilizing a number of techniques. Nucleic acid from any nucleated cell can be used as the starting point for such assay techniques, and may be isolated according to standard nucleic acid preparation procedures which are well known to those of skill in the art.

DNA may be used in hybridization or amplification assays of biological samples to detect abnormalities involving FPR class receptor gene structure, including point mutations, insertions, deletions and chromosomal rearrangements. Such assays may include, but are not limited to, Southern analyses, single stranded conformational polymorphism analyses (SSCP), and PCR analyses.

Such diagnostic methods for the detection of FPR class receptor gene-specific mutations can involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, as described in the section above, under conditions favorable for the specific annealing of these reagents to their complementary sequences within the FPR class receptor gene. Preferably, the lengths of these nucleic acid reagents are at least 15 to 30 nucleotides. After incubation, all non-annealed nucleic acids are removed from the nucleic acid:FPR class receptor molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents of the type described in the section above are easily removed. Detection of the remaining, annealed, labeled FPR class receptor nucleic acid reagents is accomplished using standard techniques well-known to those in the art. The FPR class receptor gene sequences to which the nucleic acid reagents have annealed can be compared to the annealing pattern expected from a normal FPR class receptor gene sequence in order to determine whether an FPR class receptor gene mutation is present.

Alternative diagnostic methods for the detection of FPR class receptor gene specific nucleic acid molecules, in patient samples or other appropriate cell sources, may involve their amplification, e.g., by PCR (the experimental embodiment set forth in Mullis, K. B. 1987 U.S. Pat. No. 4,683,202), followed by the detection of the amplified molecules using techniques well known to those of skill in the art. The resulting amplified sequences can be compared to those which would be expected if the nucleic acid being amplified contained only normal copies of the FPR class receptor gene in order to determine whether an FPR class receptor gene mutation exists.

Additionally, well-known genotyping techniques can be performed to identify individuals carrying FPR class receptor gene mutations. Such techniques include, for example, the use of restriction fragment length polymorphisms (RFLPs), which involve sequence variations in one of the recognition sites for the specific restriction enzyme used.

Additionally, improved methods for analyzing DNA polymorphisms which can be utilized for the identification of FPR class receptor gene mutations have been described which capitalize on the presence of variable numbers of short, tandemly repeated DNA sequences between the restriction enzyme sites. For example, Weber (U.S. Pat. No. 5,075,217, which is incorporated herein by reference in its entirety) describes a DNA marker based on length polymorphisms in blocks of (dC-dA)ₙ-(dG-dT)ₙ short tandem repeats. The average separation of (dC-dA)ₙ-(dG-dT)ₙ blocks is estimated to be 30,000-60,000 bp. Markers which are so closely spaced exhibit a high frequency co-inheritance, and are extremely useful in the identification of genetic mutations, such as, for example, mutations within the FPR class receptor gene, and the diagnosis of diseases and disorders related to FPR class receptor mutations.

Also, Caskey et al. (U.S. Pat. No. 5,364,759, which is incorporated herein by reference in its entirety) describe a DNA profiling assay for detecting short tri and tetra nucleotide repeat sequences. The process includes extracting the DNA of interest, such as the FPR class receptor gene, amplifying the extracted DNA, and labeling the repeat sequences to form a genotypic map of the individual's DNA.

The level of FPR class receptor gene expression can also be assayed by detecting and measuring FPR class receptor transcription. For example, RNA from a cell type or tissue known, or suspected to express the FPR class receptor gene, such as brain tissue, may be isolated and tested utilizing hybridization or PCR techniques such as are described above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the FPR class receptor gene. Such analyses may reveal both quantitative and qualitative aspects of the expression pattern of the FPR class receptor gene, including activation or inactivation of FPR class receptor gene expression.

In one embodiment of such a detection scheme, cDNAs are synthesized from the RNAs of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the FPR class receptor nucleic acid reagents described in the section above. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such FPR class receptor gene expression assays “in situ”, i.e., directly upon tissue sections (fixed and/or frozen) of patient tissue obtained from biopsies or resections, such that no nucleic acid purification is necessary. Nucleic acid reagents such as those described in the section above may be used as probes and/or primers for such in situ procedures (see, for example, Nuovo G. J. 1992 PCR In Situ Hybridization: Protocols And Applications Raven Press, NY).

Alternatively, if a sufficient quantity of the appropriate cells can be obtained, standard Northern analysis can be performed to determine the level of mRNA expression of the FPR class receptor gene.

### Detection of FPR Class Receptor Gene Products

Antibodies directed against wild type or mutant FPR class receptor gene products or conserved variants or peptide fragments thereof, which are discussed above, may also be used as inflammatory disorder diagnostics and prognostics, as described herein. Such diagnostic methods, may be used to detect abnormalities in the level of FPR class receptor gene expression, or abnormalities in the structure and/or temporal, tissue, cellular, or subcellular location of the FPR class receptor, and may be performed in vivo or in vitro, such as, for example, on biopsy tissue.

For example, antibodies directed to epitopes of the FPR class receptor extra-cellular domain can be used in vivo to detect the pattern and level of expression of the FPR class receptor in the body. Such antibodies can be labeled, e.g., with a radio-opaque or other appropriate compound and injected into a subject in order to visualize binding to the FPR class receptor expressed in the body using methods such as X-rays, CAT-scans, or MRI. Labeled antibody fragments, e.g., the Fab or single chain antibody comprising the smallest portion of the antigen binding region, are preferred for this purpose to promote crossing the blood-brain barrier and permit labeling FPR class receptors expressed in the brain tissue.

Additionally, any FPR class receptor fusion protein or FPR class receptor conjugated protein whose presence can be detected, can be administered. For example, FPR class receptor fusion or conjugated proteins labeled with a radio-opaque or other appropriate compound can be administered and visualized in vivo, as discussed, above for labeled antibodies. Further Aβ fusion proteins can be utilized for in vitro diagnostic procedures.

Alternatively, immunoassays or fusion protein detection assays, as described above, can be utilized on biopsy and autopsy samples in vitro to permit assessment of the expression pattern of the FPR class receptor. Such assays are not confined to the use of antibodies that define the FPR class receptor extra-cellular domain, but can include the use of antibodies directed to epitopes of any of the domains of the FPR class receptor, e.g., the extra-cellular domain, the transmembrane domain and/or cytoplasmic domain. The use of each or all of these labeled antibodies will yield useful information regarding translation and intracellular transport of the FPR class receptor to the cell surface, and can identify defects in processing.

The tissue or cell type to be analyzed will generally include those which are known, or suspected, to express the FPR class receptor gene, such as, for example, brain tissue cells. The protein isolation methods employed herein may, for example, be such as those described in Harlow and Lane (Harlow E. & Lane D. 1988 Antibodies: A Laboratory Manual Cold Spring Harbor Laboratory Press, Cold Spring Harbor, N.Y.), which is incorporated herein by reference in its entirety. The isolated cells can be derived from cell culture or from a patient. The analysis of cells taken from culture may be a necessary step in the assessment of cells that could be used as part of a cell-based gene therapy technique or, alternatively, to test the effect of compounds on the expression of the FPR class receptor gene.

For example, antibodies, or fragments of antibodies, such as those described in the section above, useful in the present invention may be used to quantitatively or qualitatively detect the presence of FPR class receptor gene products or conserved variants or peptide fragments thereof. This can be accomplished, for example, by immunofluorescence techniques employing a fluorescently labeled antibody (see below) coupled with light microscopic, flow cytometric, or fluorimetric detection. Such techniques are especially preferred if such FPR class receptor gene products are expressed on the cell surface.

The antibodies (or fragments thereof) or Aβ fusion or conjugated proteins useful in the present invention may, additionally, be employed histologically, as in immunofluorescence, immunoelectron microscopy or non-immuno assays, for in situ detection of FPR class receptor gene products or conserved variants or peptide fragments thereof, or for Aβ binding (in the case of labeled Aβ fusion protein).

In situ detection may be accomplished by removing a histological specimen from a patient, and applying thereto a labeled antibody or fusion protein of the present invention. The antibody (or fragment) or fusion protein is preferably applied by overlaying the labeled antibody (or fragment) onto a biological sample. Through the use of such a procedure, it is possible to determine not only the presence of the FPR class receptor gene product, or conserved variants or peptide fragments, or Aβ binding, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for FPR class receptor gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying FPR class receptor gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled FPR class receptor antibody or Aβ fusion protein. The solid phase support may then be washed with the buffer a second time to remove unbound antibody or fusion protein. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of FPR class receptor antibody or Aβ fusion protein may be determined according to well-known methods. Those skilled in the art will be able to determine operative and optimal assay conditions for each determination by employing routine experimentation.

With respect to antibodies, one of the ways in which the FPR class receptor antibody can be detectably labeled is by linking the same to an enzyme and use in an enzyme immunoassay (EIA) (Voller A. 1978 The Enzyme Linked Immunosorbent Assay (ELISA) Diagnostic Horizons 2:1-7, Microbiological Associates Quarterly Publication, Walkersville, Md.); Voller A. et al. 1978 J Clin Pathol 31:507-520; Butler J. E. 1981 Meth Enzymol 73:482-523; Maggio E. ed. 1980 Enzyme Immunoassay CRC Press, Boca Raton, Fla.; Ishikawa E. et al. eds. 1981 Enzyme Immunoassay Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alphaglycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect FPR class receptor through the use of a radioimmunoassay (RIA) (see, for example, Weintraub B. 1986 Principles of Radioimmunoassays, Seventh Training Course on Radioligand Assay Techniques The Endocrine Society, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### Screening Assays for Compounds that Modulate FPR Class Receptor Expression or Activity

The following assays are designed to identify compounds that interact with (e.g., bind to) FPR class receptor (including, but not limited to the extra-cellular domain or cytoplasmic domain of FPR class receptor), compounds that interact with (e.g., bind to) intracellular proteins that interact with FPR class receptor (including, but not limited to, the transmembrane and cytoplasmic domain of FPR class receptor), compounds that interfere with the interaction of FPR class receptor with transmembrane or intracellular proteins involved in FPR class receptor-mediated signal transduction, and to compounds which modulate the activity of FPR class receptor gene (i.e., modulate the level of FPR class receptor gene expression) or modulate the level of FPR class receptor. Assays may additionally be utilized which identify compounds which bind to FPR class receptor gene regulatory sequences (e.g., promoter sequences) and which may modulate FPR class receptor gene expression. See, e.g., Platt K. A. 1994 J Biol Chem 269:28558-28562, which is incorporated herein by reference in its entirety.

The compounds which may be screened in accordance with the invention include, but are not limited to peptides, antibodies and fragments thereof, and other organic compounds (e.g., peptidomimetics) that bind to the extra-cellular domain of the FPR class receptor and either mimic the activity triggered by the natural ligand (i.e., agonists) or inhibit the activity triggered by the natural ligand (i.e., antagonists); as well as peptides, antibodies or fragments thereof, and other organic compounds that mimic the extra-cellular domain of the FPR class receptor (or a portion thereof) and bind to and “neutralize” natural ligand.

Such compounds may include, but are not limited to, peptides such as, for example, soluble peptides, including but not limited to members of random peptide libraries (see, e.g., Lam K. S. et al. 1991 Nature 354:82-84; Houghten R. et al. 1991 Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang Z. et al. 1993 Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened in accordance with the invention include but are not limited to small organic molecules that are able to cross the blood-brain barrier, gain entry into an appropriate cell (e.g., in brain tissue) and affect the expression of the FPR class receptor gene or some other gene involved in the FPR class receptor signal transduction pathway (e.g., by interacting with the regulatory region or transcription factors involved in gene expression); or such compounds that affect the activity of the FPR class receptor (e.g., by inhibiting or enhancing the enzymatic activity of the cytoplasmic domain) or the activity of some other intracellular factor involved in the FPR class receptor signal transduction pathway.

Computer modeling and searching technologies permit identification of compounds, or the improvement of already identified compounds, that can modulate FPR class receptor expression or activity. Having identified such a compound or composition, the active sites or regions are identified. Such active sites might typically be ligand binding sites, such as the interaction domains of Aβ with FPR class receptor itself. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential FPR class receptor modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of Aβ, FPR class receptor, and related transduction and transcription factors will be apparent to those of skill in the art.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen et al. 1988 Acta Pharmaceutical Fennica 97:159-166; Ripka 1998 New Scientist 54-57; McKinaly & Rossmann 1989 Annu Rev Pharmacol Toxicol 29:111-122; Perry & Davies 1989 OSAR: Quantitative Structure-Activity Relationships in Drug Design pp. 189-193 Alan R. Liss, Inc.; Lewis & Dean 1989 Proc R Soc Lond 236:125-140 and 141-162; and, with respect to a model receptor for nucleic acid components, Askew et al. 1989 J Am Chem Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elaborating the biological function of the FPR class receptor gene product, and for ameliorating inflammation disorders, including inflammation in AD. Assays for testing the effectiveness of compounds, identified by, for example, techniques such as those described hereinbelow, are discussed in the sections infra.

### In Vitro Screening Assays for Compounds that Bind to FRP Class Receptor

In vitro systems may be designed to identify compounds capable of interacting with (e.g., binding to) FPR class receptor (including, but not limited to, the extra-cellular domain or cytoplasmic domain of FPR class receptor). Compounds identified may be useful, for example, in modulating the activity of wild type and/or mutant FPR class receptor gene products; may be useful in elaborating the biological function of the FPR class receptor; may be utilized in screens for identifying compounds that disrupt normal FPR class receptor interactions; or may in themselves disrupt such interactions.

The principle of the assays used to identify compounds that bind to the FPR class receptor involves preparing a reaction mixture of the FPR class receptor and the test compound under conditions and for a time sufficient to allow the two components to interact and bind, thus forming a complex which can be removed and/or detected in the reaction mixture. The FPR class receptor species used can vary depending upon the goal of the screening assay. For example, where agonists of the natural ligand are sought, the full length FPR class receptor, or a soluble truncated FPR class receptor, e.g., in which the transmembrane domain and/or cytoplasmic domain is deleted from the molecule, a peptide corresponding to the extracellular domain or a fusion protein containing the FPR class receptor extracellular domain fused to a protein or polypeptide that affords advantages in the assay system (e.g., labeling, isolation of the resulting complex, etc.) can be utilized. Where compounds that interact with the cytoplasmic domain are sought to be identified, peptides corresponding to the FPR class receptor cytoplasmic domain and fusion proteins containing the FPR class receptor cytoplasmic domain can be used.

The screening assays can be conducted in a variety of ways. For example, one method to conduct such an assay would involve anchoring the FPR class receptor protein, polypeptide, peptide or fusion protein or the test substance onto a solid phase and detecting FPR class receptor/test compound complexes anchored on the solid phase at the end of the reaction. In one embodiment of such a method, the FPR class receptor reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Other solid phase supports include, but are not limited to, the walls of wells of a reaction tray, test tubes, polystyrene beads, magnetic beads, nitrocellulose strips, membranes, microparticles such as latex particles, animal cells, Duracyte®, artificial cells, and others. The anchored component can also be joined to inorganic carriers, such as silicon oxide material (e.g. silica gel, zeolite, diatomaceous earth or aminated glass) by, for example, a covalent linkage through a hydroxy, carboxy or amino group and a reactive group on the carrier. Additionally, the anchored component can be covalently bound to proteins and oligo/polysaccharides (e.g. cellulose, starch, glycogen, chitosan or aminated sepharose) by utilizing a reactive group on the molecule, such as a hydroxy or an amino group. Further, supports having other reactive groups that are chemically activated so as to attach the anchored component can be used. For example, cyanogen bromide activated matrices, epoxy activated matrices, thio and thiopropyl gels, nitrophenyl chloroformate and N-hydroxy succinimide chloroformate linkages, or oxirane acrylic supports are used (Sigma). Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for FPR class receptor protein, polypeptide, peptide or fusion protein or the test compound to anchor any complexes formed in solution, and a labeled antibody specific for the other component of the possible complex to detect anchored complexes.

Alternatively, cell-based assays can be used to identify compounds that interact with FPR class receptor. To this end, cell lines that express FPR class receptor, or cell lines (e.g., COS cells, CHO cells, fibroblasts, etc.) that have been genetically engineered to express FPR class receptor (e.g., by transfection or transduction of FPR class receptor DNA) can be used. Interaction of the test compound with, for example, the extra-cellular domain of FPR class receptor expressed by the host cell can be determined by comparison or competition with native Aβ.

### Assays for Intracellular Proteins that Interact with FPR Class Receptor

Any method suitable for detecting protein-protein interactions may be employed for identifying transmembrane proteins or intracellular proteins that interact with FPR class receptor. Among the traditional methods which may be employed are co-immunoprecipitation, crosslinking and co-purification through gradients or chromatographic columns of cell lysates or proteins obtained from cell lysates and the FPR class receptor to identify proteins in the lysate that interact with the FPR class receptor. For these assays, the FPR class receptor component used can be a full length FPR class receptor, a soluble derivative lacking the membrane-anchoring region (e.g., a truncated FPR class receptor in which the transmembrane is deleted resulting in a truncated molecule containing the extra-cellular domain fused to the cytoplasmic domain), a peptide corresponding to the cytoplasmic domain or a fusion protein containing the cytoplasmic domain of FPR class receptor. Once isolated, such an intracellular protein can be identified and can, in turn, be used, in conjunction with standard techniques, to identify proteins with which it interacts. For example, at least a portion of the amino acid sequence of an intracellular protein which interacts with the FPR class receptor can be ascertained using techniques well known to those of skill in the art, such as via the Edman degradation technique (see, e.g., Creighton 1983 Proteins: Structures and Molecular Principles W. H. Freeman & Co., N.Y., pp. 34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well known.\ (See, e.g., Ausubel et al. 1989 Current Protocols in Molecular Biology, Green Publishing Associates and Wiley Interscience, N.Y.; and Innis M. et al. eds. 1990 PCR Protocols: A Guide to Methods and Applications Academic Press, Inc., New York).

Additionally, methods may be employed which result in the simultaneous identification of genes which encode the transmembrane or intracellular proteins interacting with FPR class receptor. These methods include, for example, probing expression libraries, in a manner similar to the well known technique of antibody probing of λgt11 libraries, using labeled FPR class receptor protein, or an FPR class receptor polypeptide, peptide or fusion protein, e.g., an FPR class receptor polypeptide or FPR class receptor domain fused to a marker (e.g., an enzyme, fluor, luminescent protein, or dye), or an Ig-Fc domain.

One method which detects protein interactions in vivo, the two-hybrid system, is described in detail for illustration only and not by way of limitation. One version of this system has been described (Chien et al. 1991 PNAS USA 8:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to an FPR class receptor nucleotide sequence encoding FPR class receptor, an FPR class receptor polypeptide, peptide or fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, FPR class receptor may be used as the bait gene product. Total genomic or cDNA sequences are fused to the DNA encoding an activation domain. This library and a plasmid encoding a hybrid of a bait FPR class receptor gene product fused to the DNA-binding domain are cotransformed into a yeast reporter strain, and the resulting transformants are screened for those that express the reporter gene. For example, and not by way of limitation, a bait FPR class receptor gene sequence, such as the open reading frame of FPR class receptor (or a domain of FPR class receptor) can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait FPR class receptor gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait FPR class receptor gene-GAL4 fusion plasmid into a yeast strain which contains a lacZ gene driven by a promoter which contains GAL4 activation sequence. A cDNA encoded protein, fused to GAL4 transcriptional activation domain, that interacts with bait FPR class receptor gene product will reconstitute an active GAL4 protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait FPR class receptor gene product-interacting protein using techniques routinely practiced in the art.

### Assays for Compounds that Interfere with FPR class receptor/Extra-cellular, FPR Class Receptor/Intra-cellular or FPR Class Receptor/Transmembrane Macromolecule Interaction

The macromolecules that interact with the FPR class receptor are referred to, for purposes of this discussion, as “binding partners”. These binding partners, such as Aβ, are involved in the FPR class receptor signal transduction pathway, and therefore, in the role of FPR class receptor in inflammation, including inflammation in AD. Therefore, it is desirable to identify compounds that interfere with or disrupt the interaction of such binding partners with Aβ which may be useful in regulating the activity of the FPR class receptor and control inflammatory disorders associated with FPR class receptor activity, including inflammation in AD.

The basic principle of the assay systems used to identify compounds that interfere with the interaction between the FPR class receptor and its binding partner or partners involves preparing a reaction mixture containing FPR class receptor protein, polypeptide, peptide or fusion protein as described in the sections above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the FPR class receptor moiety and its binding partner. Control reaction mixtures are incubated without the test compound or with a placebo. The formation of any complexes between the FPR class receptor moiety and the binding partner is then detected. The formation of a complex in the control reaction, but not in the reaction mixture containing the test compound, indicates that the compound interferes with the interaction of the FPR class receptor and the interactive binding partner. Additionally, complex formation within reaction mixtures containing the test compound and normal FPR class receptor protein may also be compared to complex formation within reaction mixtures containing the test compound and a mutant FPR class receptor. This comparison may be important in those cases wherein it is desirable to identify compounds that disrupt interactions of mutant but not normal FPR class receptors.

The assay for compounds that interfere with the interaction of the FPR class receptor and binding partners can be conducted in a heterogeneous or homogeneous format. Heterogeneous assays involve anchoring either the FPR class receptor moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the FPR class receptor moiety and interactive binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the FPR class receptor moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the FPR class receptor gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the FPR class receptor moiety and the interactive binding partner is prepared in which either the FPR class receptor or its binding partners is labeled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt FPR class receptor/intracellular binding partner interaction can be identified.

In a particular embodiment, an FPR class receptor fusion can be prepared for immobilization. For example, the FPR class receptor or a peptide fragment, e.g., corresponding to the extra-cellular domain or the cytoplasmic domain, can be fused to a glutathione-S-transferase (GST) gene using a fusion vector, such as pGEX-5X-1, in such a manner that its binding activity is maintained in the resulting fusion protein. The interactive binding partner can be purified and used to prepare a monoclonal antibody, using methods routinely practiced in the art and described above. This antibody can be labeled with the radioactive isotope ¹²⁵I, for example, by methods routinely practiced in the art. In a heterogeneous assay, e.g., the GST-FPR class receptor fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the FPR class receptor gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-FPR class receptor fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the FPR class receptor/binding partner interaction can be detected by adding the labeled antibody and measuring the radioactivity associated with the beads.

In another embodiment of the invention, these same techniques can be employed using peptide fragments that correspond to the binding domains of the FPR class receptor and/or the interactive binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the binding partner is obtained, short gene segments can be engineered to express peptide fragments of the protein, which can then be tested for binding activity and purified or synthesized.

For example, and not by way of limitation, an FPR class receptor gene product can be anchored to a solid material as described, above, by making a GST-FPR class receptor fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-FPR class receptor fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

### Assays for Identification of Compounds that Ameliorate Inflammation, Including Inflammation in AD

Compounds, including but not limited to binding compounds identified via assay techniques such as those described hereinabove can be tested for the ability to inhibit inflammation, including inflammation in AD. The assays described above can identify compounds which affect FPR class receptor activity (e.g., compounds that bind to the FPR class receptor, inhibit binding of the natural ligand, and either activate signal transduction (agonists) or block activation (antagonists), and compounds that bind to the natural ligand of the FPR class receptor and neutralize ligand activity); or compounds that affect FPR class receptor gene activity (by affecting FPR class receptor gene expression, including molecules, e.g., proteins or small organic molecules, that affect or interfere with splicing events so that expression of the full length FPR class receptor can be modulated). However, it should be noted that the assays described can also identify compounds that modulate FPR class receptor signal transduction (e.g., compounds which affect downstream signalling events, such as inhibitors or enhancers of tyrosine kinase or phosphatase activities which participate in transducing the signal activated by Aβ binding to the FPR class receptor). The identification and use of such compounds which affect another step in the FPR class receptor signal transduction pathway in which the FPR class receptor gene and/or FPR class receptor gene product is involved and, by affecting this same pathway may modulate the effect of FPR class receptor on the development of inflammation, including inflammation in AD, are within the scope of the invention. Such compounds can be used as part of a therapeutic method for the treatment of inflammation, including inflammation in AD.

The invention encompasses cell-based and animal model-based assays for the identification of compounds exhibiting such an ability to ameliorate symptoms associated with inflammation, including inflammation in AD. Such cell-based assay systems can also be used as the “gold standard” to assay for purity and potency of the natural ligand, Aβ, including recombinantly or synthetically produced Aβ and Aβ mutants.

Cell-based systems can be used to identify compounds which may act to ameliorate symptoms of inflammation, including inflammation in AD. Such cell systems can include, for example, recombinant or non-recombinant cells, such as cell lines, which express the FPR class receptor gene. For example brain tissue cells or cell lines derived from brain tissue can be used. In addition, expression host cells (e.g., COS cells, CHO cells, fibroblasts) genetically engineered to express a functional FPR class receptor and to respond to activation by the natural Aβ ligand, e.g., as measured by a chemical or phenotypic change, induction of another host cell gene, change in ion flux (e.g., Ca²⁺), tyrosine phosphorylation of host cell proteins, etc., can be used as an end point in the assay.

In utilizing such cell systems, cells may be exposed to a compound suspected of exhibiting an ability to ameliorate symptoms of inflammation, including inflammation in AD, at a sufficient concentration and for a time sufficient to elicit such an amelioration of alterations associated with such symptoms in the exposed cells. After exposure, the cells can be assayed to measure alterations in the expression of the FPR class receptor gene, e.g., by assaying cell lysates for FPR class receptor mRNA transcripts (e.g., by Northern analysis) or for FPR class receptor protein expressed in the cell; compounds which regulate or modulate expression of the FPR class receptor gene are good candidates as therapeutics. Alternatively, the cells are examined to determine whether one or more cellular phenotypes has been altered to resemble a more normal or more wild type, non-disorder phenotype, or a phenotype more likely to produce a lower incidence or severity of disorder symptoms. Still further, the expression and/or activity of components of the signal transduction pathway of which FPR class receptor is a part, or the activity of the FPR class receptor signal transduction pathway itself can be assayed.

For example, after exposure, the cell lysates can be assayed for the presence of tyrosine phosphorylation of host cell proteins, as compared to lysates derived from unexposed control cells. The ability of a test compound to inhibit tyrosine phosphorylation of host cell proteins in these assay systems indicates that the test compound inhibits signal transduction initiated by FPR class receptor activation. The cell lysates can be readily assayed using a Western blot format; i.e., the host cell proteins are resolved by gel electrophoresis, transferred and probed using a anti-phosphotyrosine detection antibody (e.g., an anti-phosphotyrosine antibody labeled with a signal generating compound, such as radiolabel, fluor, enzyme, etc.) (see, e.g., Glenney et al. 1988 J Immunol Methods 109:277-285; Frackelton et al. 1983 Mol Cell Biol 3:1343-1352). Alternatively, an ELISA format could be used in which a particular host cell protein involved in the FPR class receptor signal transduction pathway is immobilized using an anchoring antibody specific for the target host cell protein, and the presence or absence of phosphotyrosine on the immobilized host cell protein is detected using a labeled anti-phosphotyrosine antibody (see, King et al. 1993 Life Sciences 53:1465-1472). In yet another approach, ion flux, such as calcium flux, can be measured as an end point for FPR class receptor stimulated signal transduction.

In addition, animal-based systems, which may include, for example, transgenic mice, may be used to identify compounds capable of ameliorating symptoms associated with inflammation, including inflammation in AD. Such animal models may be used as test substrates for the identification of drugs, pharmaceuticals, therapies and interventions which may be effective in treating such disorders. For example, animal models may be exposed to a compound, suspected of exhibiting an ability to ameliorate symptoms associated with inflammation, including inflammation in AD, at a sufficient concentration and for a time sufficient to elicit such an amelioration of symptoms in the exposed animals. The response of the animals to the exposure may be monitored by assessing the reversal of disorders associated with inflammation, including inflammation in AD. With regard to intervention, any treatments which reverse any aspect of symptoms associated with inflammation, including inflammation in AD, should be considered as candidates for human therapeutic intervention. Dosages of test agents may be determined by deriving dose-response curves, as discussed in the section below.

### The Treatment of Inflammation, Including Inflammation in AD

The invention encompasses methods and compositions for modifying inflammation, including inflammation in AD. Symptoms of inflammation, including inflammation in AD, may be ameliorated by decreasing the level of FPR class receptor gene expression, and/or FPR class receptor gene activity, and/or downregulating activity of the FPR class receptor pathway (e.g., by targeting downstream signalling events). Different approaches are discussed below.

### Inhibition of FPR Class Receptor Expression or FPR Class Receptor Activity to Ameliorate Inflammation, Including Inflammation in AD

Any method which neutralizes Aβ or inhibits expression of the FPR class receptor gene (either transcription or translation) can be used to ameliorate inflammation, including inflammation in AD.

For example, the administration of soluble peptides, proteins, fusion proteins, or antibodies (including anti-idiotypic antibodies) that bind to and “neutralize” circulating Aβ, the natural ligand for the FPR class receptor, can be used to ameliorate inflammation, including inflammation in AD. To this end, peptides corresponding to the extra-cellular domain of FPR class receptor, soluble deletion mutants of FPR class receptor (mutants lacking the transmembrane or cytoplasmic domain), or either of these FPR class receptor domains or mutants fused to another polypeptide (e.g., an IgFc polypeptide) can be utilized. Alternatively, anti-idiotypic antibodies or Fab fragments of antiidiotypic antibodies that mimic the FPR class receptor extra-cellular domain and neutralize Aβ can be used (see supra). Such FPR class receptor peptides, proteins, fusion proteins, anti-idiotypic antibodies or Fabs are administered to a subject in amounts sufficient to neutralize Aβ and to ameliorate inflammation, including inflammation in AD.

FPR class receptor peptides corresponding to the extra-cellular domain can be used. FPR class receptor transmembrane mutants in which all or part of the hydrophobic anchor sequence could also be used. Fusion of the FPR class receptor, the FPR class receptor extra-cellular domain or the FPR class receptor transmembrane mutants to an IgFc polypeptide should not only increase the stability of the preparation, but will increase the half-life and activity of the FPR class receptor-Ig fusion protein in vivo. The Fc region of the Ig portion of the fusion protein may be further modified to reduce immunoglobulin effector function.

In an alternative embodiment for neutralizing circulating Aβ, cells that are genetically engineered to express such soluble or secreted forms of FPR class receptor may be administered to a patient, whereupon they will serve as “bioreactors” in vivo to provide a continuous supply of the Aβ neutralizing protein. Such cells may be obtained from the patient or an MHC compatible donor and can include, but are not limited to fibroblasts, blood cells (e.g., lymphocytes), adipocytes, muscle cells, endothelial cells, etc. The cells are genetically engineered in vitro using recombinant DNA techniques to introduce the coding sequence for the FPR class receptor extra-cellular domain, FPR class receptor transmembrane mutants, or for FPR class receptor-Ig fusion protein into the cells, e.g., by transduction (using viral vectors, and preferably vectors that integrate the transgene into the cell genome) or transfection procedures, including but not limited to the use of plasmids, cosmids, YACs, electroporation, liposomes, etc. The FPR class receptor coding sequence can be placed under the control of a strong constitutive or inducible promoter or promoter/enhancer to achieve expression and secretion of the FPR class receptor peptide or fusion protein. The engineered cells which express and secrete the desired FPR class receptor product can be introduced into the patient systemically, e.g., in the circulation, intraperitoneally, into the brain tissue. Alternatively, the cells can be incorporated into a matrix and implanted in the body, e.g., genetically engineered fibroblasts can be implanted as part of a skin graft; genetically engineered endothelial cells can be implanted as part of a vascular graft (see, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous cells, they can be administered using well-known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

In an alternate embodiment, therapy for inflammation, including inflammation in AD, can be designed to reduce the level of endogenous FPR class receptor gene expression, e.g., using antisense or ribozyme approaches to inhibit or prevent translation of FPR class receptor mRNA transcripts; triple helix approaches to inhibit transcription of the FPR class receptor gene; or targeted homologous recombination to inactivate or “knock out” the FPR class receptor gene or its endogenous promoter. Because the FPR class receptor gene is expressed in the brain, delivery techniques should be preferably designed to cross the blood-brain barrier (see PCT WO89/10134, which is incorporated by reference herein in its entirety). Alternatively, the antisense, ribozyme or DNA constructs described herein could be administered directly to the site containing the target cells, e.g., the brain tissue.

Antisense approaches involve the design of oligonucleotides (either DNA or RNA) that are complementary to FPR class receptor mRNA. The antisense oligonucleotides will bind to the complementary FPR class receptor mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have shown to be effective at inhibiting translation of mRNAs as well (see generally, Wagner, R. 1994 Nature 372:333-335). Thus, oligonucleotides complementary to either the 5′- or 3′-non-translated, non-coding regions of the FPR class receptor could be used in an antisense approach to inhibit translation of endogenous FPR class receptor mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of FPR class receptor mRNA, antisense nucleic acids should be at least six nucleotides in length, and are preferably oligonucleotides ranging from 6 to about 50 nucleotides in length. In specific aspects the oligonucleotide is at least 13 nucleotides, at least 17 nucleotides, at least 25 nucleotides, or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo); or agents facilitating transport across the cell membrane (see, e.g., Letsinger et al. 1989 PNAS USA 86:6553-6556; Lemaitre et al. 1987 PNAS USA 84:648-652; PCT Publication No. WO88/09810, published Dec. 15, 1988), or the blood-brain barrier (see, e.g., PCT Publication No. WO89/10134, published Apr. 25, 1988); hybridization-triggered cleavage agents (see, e.g., Krol et al. 1988 BioTechniques 6:958-976); or intercalating agents (see, e.g., Zon 1988 Pharm Res 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al. 1987 Nucl Acids Res 15:6625-6641). The oligonucleotide is a 2′-β-methylribonucleotide (Inoue et al. 1987 Nucl Acids Res 15:6131-6148), or a chimeric RNA-DNA analogue (Inoue et al. 1987 FEBS Lett 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988 Nucl Acids Res 16:3209), methylphosphonate oligonucleotides can be prepared by use of controlled pore glass polymer supports (Sarin et al. 1988 PNAS USA 85:7448-7451), etc.

The antisense molecules should be delivered to cells which express the FPR class receptor in vivo, e.g., brain tissue. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous FPR class receptor transcripts and thereby prevent translation of the FPR class receptor mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981 Nature 290:304-310), the promoter contained in the 3′ long terminal repeat of Rous sarcoma virus (Yamamoto et al. 1980 Cell 22:787-797), the herpes thymidine kinase promoter (Wagner et al. 1981 PNAS USA 78:1441-1445), the regulatory sequences of the metallothionein gene (Brinster et al. 1982 Nature 296:39-42), etc. Any type of plasmid, cosmid, YAC or viral vector can be used to prepare the recombinant DNA construct which can be introduced directly into the tissue site, e.g., brain tissue. Alternatively, viral vectors can be used which selectively infect the desired tissue, (e.g., for brain, herpesvirus vectors may be used), in which case administration may be accomplished by another route (e.g., systemically).

Ribozyme molecules designed to catalytically cleave FPR class receptor mRNA transcripts can also be used to prevent translation of FPR class receptor mRNA and expression of FPR class receptor (see, e.g., PCT International Publication WO90/11364, published Oct. 4, 1990; Sarver et al. 1990 Science 247:1222-1225). While ribozymes that cleave mRNA at site-specific recognition sequences can be used to destroy FPR class receptor mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach 1988 Nature 334:585-591. There are presumably many potential hammerhead ribozyme cleavage sites within the nucleotide sequence of human FPR class receptor cDNA. Preferably the ribozyme is engineered so that the cleavage recognition site is located near the 5′ tend of the FPR class receptor mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug et al. 1984 Science 224:574-578; Zaug & Cech 1986 Science 231:470-475; Zaug et al. 1986 Nature 324:429-433; published International patent-application No. WO 88/04300 by University Patents Inc.; Been & Cech 1986 Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in FPR class receptor.

As in the antisense approach, the ribozymes can be composed of modified oligonucleotides (e.g., for improved stability, targeting, etc.) and should be delivered to cells which express the FPR class receptor in vivo, e.g., brain tissue. A preferred method of delivery involves using a DNA construct “encoding” the ribozyme under the control of a strong constitutive pol III or pol II promoter, so that transfected cells will produce sufficient quantities of the ribozyme to destroy endogenous FPR class receptor messages and inhibit translation. Because ribozymes unlike antisense molecules, are catalytic, a lower intracellular concentration is required for efficiency.

Endogenous FPR class receptor gene expression can also be reduced by inactivating or “knocking out” the FPR class receptor gene or its promoter using targeted homologous recombination (e.g., see Smithies et al. 1985 Nature 317:230-234; Thomas & Capecchi 1987 Cell 51:503-512; Thompson et al. 1989 Cell 5:313-321; each of which is incorporated by reference herein in its entirety). For example, a mutant, non-functional FPR class receptor (or a completely unrelated DNA sequence) flanked by DNA homologous to the endogenous FPR class receptor gene (either the coding regions or regulatory regions of the FPR class receptor gene) can be used, with or without a selectable marker and/or a negative selectable marker, to transfect cells that express FPR class receptor in vivo. Insertion of the DNA construct, via targeted homologous recombination, results in inactivation of the FPR class receptor gene. Such approaches are particularly suited in the agricultural field where modifications to ES (embryonic stem) cells can be used to generate animal offspring with an inactive FPR class receptor (e.g., see Thomas & Capecchi 1987 Cell 51:503-512; Thompson et al. 1989 Cell 5:313-321). However this approach can be adapted for use in humans provided the recombinant DNA constructs are directly administered or targeted to the required site in vivo using appropriate viral vectors, e.g., herpes virus vectors for delivery to brain tissue.

Alternatively, endogenous FPR class receptor gene expression can be reduced by targeting deoxyribonucleotide sequences complementary to the regulatory region of the FPR class receptor gene (i.e., the FPR class receptor promoter and/or enhancers) to form triple helical structures that prevent transcription of the FPR class receptor gene in target cells in the body (see generally, Helene C. 1991 Anticancer Drug Des 6:569-84; Helene C. et al. 1992 Ann NY Acad Sci 660:27-36; and Maher L. J. 1992 Bioassays 14:807-15).

In yet another embodiment of the invention, the activity of FPR class receptor can be reduced using a “dominant negative” approach to ameliorate inflammation, including inflammation in AD. To this end, constructs which encode defective FPR class receptors can be used in gene therapy approaches to diminish the activity of the FPR class receptor in appropriate target cells. For example, nucleotide sequences that direct host cell expression of FPR class receptors in which the cytoplasmic domain or a portion of the cytoplasmic domain is deleted or mutated can be introduced into cells in brain tissue (either by in vivo or ex vivo gene therapy methods described above). Alternatively, targeted homologous recombination can be utilized to introduce such deletions or mutations into the subject's endogenous FPR class receptor gene in brain tissue. The engineered cells will express non-functional receptors (i.e., an anchored receptor that is capable of binding its natural ligand, but incapable of signal transduction). Such engineered cells present in brain tissue should demonstrate a diminished response to the endogenous Aβ ligand, resulting in amelioration of inflammation, including inflammation in AD.

### Restoration or Increase in FPR Class Receptor Expression or Activity

With respect to an increase in the level of normal FPR class receptor gene expression and/or FPR class receptor gene product activity, FPR class receptor nucleic acid sequences can be utilized for the treatment of inflammation, including inflammation in AD. Where the cause of inflammation, including inflammation in AD, is a polymorphic, defective FPR class receptor, treatment can be administered, for example, in the form of gene replacement therapy. Specifically, one or more copies of a normal FPR class receptor gene or a portion of the FPR class receptor gene that directs the production of an FPR class receptor gene product exhibiting normal function, may be inserted into the appropriate cells within a patient or animal subject, using vectors which include, but are not limited to adenovirus, adeno-associated virus, retrovirus and herpes virus vectors, in addition to other particles that introduce DNA into cells, such as liposomes.

Because the FPR class receptor gene is expressed in the brain, such gene replacement therapy techniques should be capable of delivering FPR class receptor gene sequences to these cell types within patients. Thus, the techniques for delivery of the FPR class receptor gene sequences should be designed to readily cross the blood-brain barrier, which are well known to those of skill in the art (see, e.g., PCT application, publication No. WO89/10134, which is incorporated herein by reference in its entirety), or, alternatively, should involve direct administration of such FPR class receptor gene sequences to the site of the cells in which the FPR class receptor gene sequences are to be expressed. Alternatively, targeted homologous recombination can be utilized to correct the polymorphic, defective endogenous FPR class receptor gene in the appropriate tissue; e.g., brain tissue.

Additional methods which may be utilized to increase the overall level of FPR class receptor gene expression and/or FPR class receptor activity include the introduction of appropriate FPR class receptor-expressing cells, preferably autologous cells, into a patient at positions and in numbers which are sufficient to effectuate inflammation. Such cells may be either recombinant or non-recombinant. Among the cells which can be administered to increase the overall level of FPR class receptor gene expression in a patient are normal cells, preferably brain tissue cells which express the FPR class receptor gene. The cells can be administered at the anatomical site in the brain, or as part of a tissue graft located at a different site in the body. Such cell-based gene therapy techniques are well known to those skilled in the art, see, e.g., Anderson et al. U.S. Pat. No. 5,399,349; Mulligan & Wilson, U.S. Pat. No. 5,460,959.

Finally, compounds, identified in the assays described above, that stimulate or enhance the signal transduced by activated FPR class receptor, e.g., by activating downstream signalling proteins in the FPR class receptor cascade and thereby by-passing the polymorphic, defective FPR class receptor, can be used to effectuate inflammation. The formulation and mode of administration will depend upon the physico-chemical properties of the compound. The administration should include known techniques that allow for a crossing of the blood-brain barrier.

### Pharmaceutical Preparations and Methods of Administration

The compounds that are determined to affect FPR class receptor gene expression or FPR class receptor activity can be administered to a patient at therapeutically effective doses to treat or ameliorate inflammation, including inflammation in AD. A therapeutically effective dose refers to that amount of the compound sufficient to result in amelioration of symptoms associated with inflammation, including inflammation in AD.

### Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### EXAMPLE 1

### Amyloid β₄₂ Activates a G-Protein-Coupled Chemoattractant Receptor, FPR-Like-1

Amyloid β (Aβ) is a major contributor to the pathogenesis of Alzheimer's disease (AD). Although Aβ has been reported to be directly neurotoxic, it also causes indirect neuronal damage by activating mononuclear phagocytes (microglia) that accumulate in and around senile plaques. In this study, we show that the 42 amino acid form of β amyloid peptide, Aβ₄₂, is a chemotactic agonist for a seven-transmembrane, G-protein-coupled receptor named FPR-Like-1 (FPRL1), which is expressed on human mononuclear phagocytes. Moreover, FPRL1 is expressed at high levels by inflammatory cells infiltrating senile plaques in brain tissues from AD patients. Thus, FPRL1 is envisioned as mediating inflammation seen in AD and is considered a target for developing therapeutic agents.

### Introduction

Amyloid β (Aβ) peptides play an important role in the neurodegeneration of Alzheimer's disease (AD). Mutations in the amyloid precursor protein and the presenilin genes are associated with increased production of a 42 amino acid polypeptide (Aβ₄₂) and are linked with exacerbated familial forms of AD (Selkoe D. J. 1999 Nature 399 Suppl:A23-A31). Although a direct neurotoxic effect of Aβ has been proposed (Du Yan S. et al. 1997 PNAS USA 94:5296-5301; Lambert M. P. et al. 1998 PNAS USA 95:6448-6453), the bulk of evidence favors an “indirect” pathway, based on induction by Aβ of inflammatory responses of microglia, the brain counterpart of the mononuclear phagocytes (Kalaria R. N. 1999 Curr Opin Hematol 6:15-24; Neuroinflammatory Working Group 2000 Neurobiol Aging 21:383-421). Consistent with this, activated microglia migrate to accumulate in and around the senile plaques in AD and release neurotoxic mediators in response to Aβ in vitro (Davis J. B. et al. 1992 Biochem Biophys Res Commun 189:1096-1100; London J. A. et al. 1996 PNAS USA 93:4147-4152; Meda L. et al. 1996 J Immunol 157:1213-1218; Klegeris A. & McGeer P. L. 1997 J Neurosci Res 49:229-235). Clear-cut evidence of infiltration of AD-like plaques by microglia was seen in transgenic mice overexpressing human β amyloid in the brain (Stalder M. et al. 1999 Am J Pathol 154:1673-1684). Moreover, subjects receiving anti-inflammatory drugs showed significantly delayed development of AD dementia (Kalaria R. N. 1999 Curr Opin Hematol 6:15-24; Neuroinflammatory Working Group 2000 Neurobiol Aging 21:383-421). The importance of Aβ in AD pathogenesis was further substantiated by the fact that vaccination with Aβ₄₂ of PDAPP mice, which overexpress human β amyloid in the brain, attenuated the progression of AD-like lesions (Schenk D. et al. 1999 Nature 400:173-177). Searches for a cellular receptor or receptors yielded several molecules that interact with Aβ. The scavenger receptor (SR) and the receptor for advanced glycation end products (RAGE) (El Khoury J. et al. 1996 Nature 382:716-719; Yan S. D. et al. 1996 Nature 382:685-691) bind Aβ, however, it is controversial whether they mediate a proinflammatory microglial cell response to Aβ. The existence of other functional Aβ receptor or receptors on the cell surface has been suggested (London J. A. et al. 1996 PNAS USA 93:4147-4152; Liu Y. et al. 1997 Biochem Biophys Res Commun 237:37-40; McDonald D. R. et al. 1997 J Neurosci 17:2284-2294; McDonald D. R. et al. 1998 J Neurosci 18:4451-4460; Huang F. et al. 1999 Am J Pathol 155:1741-1747). In this study, we report that a G-protein-coupled seven-transmembrane (STM) receptor, FPR-Like-1 (FPRL1), is used by Aβ₄₂ to induce migration and activation of human monocytes. We propose that FPRL1 serves as a receptor mediating the proinflammatory responses elicited by Aβ₄₂.

Reagents and cells. Aβ peptide (Aβ₄₂) and the peptide with reversed sequence (Aβ₄₂₋₁) were purchased from California Peptide Research (Napa, Calif.). All peptides were examined for endotoxin contamination and were negative at highest concentrations used in the study. Human peripheral blood monocytes were isolated from buffy coats (National Institutes of Health Clinical Center, Bethesda, Md.) enriched for mononuclear cells by using iso-osmotic Percoll gradient. The purity of the cell preparations was examined by morphology and was >90%. Rat basophilic leukemia cell line (RBL-2H3) transfected with epitope-tagged N-formylpeptide receptor (FPR) (designated ETFR) was a kind gift of Dr. R. Snyderman (Duke University, Durham, N.C.). cDNA cloning and establishment of FPRL1-transfected human embryonic kidney (HEK) 293 cells (FPRL1/293) were described previously (Gao J. L. & Murphy P. M. 1993 J Biol Chem 268:25395-25361). All the transfected cells were maintained in culture media as described (Su S. B. et al. 1999 J Exp Med 189:395-402).

Chemotaxis assays and measurement of calcium mobilization. Chemotaxis assays were performed using 48-well chemotaxis chambers (Deng X. et al. 1999 Blood 94:1165-1673). The results were expressed as the mean number (±SD) of migrated cells in three high-powered fields in three replicate samples. Chemotaxis index, which represented the fold increase in the number of cells migrated in response to chemoattractants over the cell response to control medium, also was used. Ca⁺ mobilization was measured by stimulating fura-2 AM-loaded human monocytes or receptor-transfected cells with various agents (Deng X. et al. 1999 Blood 94:1165-1673; Su S. B. et al. 1999 J Exp Med 189:395-402) and recording the ratio of fluorescence at 340 and 380 nm in a luminescence spectrometer with FL WinLab program (Perkin-Elmer, Beaconsfield, UK).

In situ hybridization. Twenty micrometer serial cryostat sections were prepared from frozen AD or normal brain tissues and mounted on glass slides. The sections were fixed in paraformaldehyde-PBS solution, washed with PBS, then acetylated in 0.25% acetic anhydride. After washing with PBS, slides were prehybridized at room temperature (RT) for 2 hr with hybridization solution (50% formamide, 5×SSC, 5× Denhardt's solution, 250 μg/ml Torula's yeast RNA, and 500 μg/ml herring sperm DNA). Hybridization was performed with digoxigenin-labeled FPRL1 cRNA probe (400 ng/ml). After overnight hybridization at 70° C., slides were washed in 0.2×SSC for 3 hr at 70° C. Anti-digoxigenin antibody conjugated with Aβ (1:2000 dilution) was applied in buffer B (0.1 M Tris-HCl, pH 7.5, and 0.15 M NaCl) containing 1% heat-inactivated goat serum and incubated overnight at RT. After extensive washing in buffer B, phosphatase reaction was performed for 3 hr in buffer C (0.1 M Tris-HCl, pH 9.5, 0.15 M NaCl, and 50 mM MgCl₂) supplemented with 0.34 mg/ml nitro blue tetrazolium, 0.23 mg/ml 5-bromo-4-chloro-3-indolyl phosphate, and 0.24 mg/ml Levamisole.

Immunohistochemistry and Congo Red staining. Serial sections of the brain tissues were fixed and incubated for 30 min with 0.3% H₂O₂, followed by 0.05% Tween 20 for 30 min and blocking serum for 60 min. The sections were reacted for 60 min at room temperature with anti-CD11b (Mac-1) antibody (1:1000) (PharMingen, San Diego, Calif.). The avidin-biotin-peroxidase method (Vector Laboratories, Burlingame, Calif.) with diaminobenzidine as the chromogen was used to visualize the antibody staining (brown products). Congo Red staining was performed on Mac-1-stained sections according to standard protocols.

Statistical analysis. All experiments were performed at least three times. The significance of the difference between test and control groups was analyzed with Student's t test.

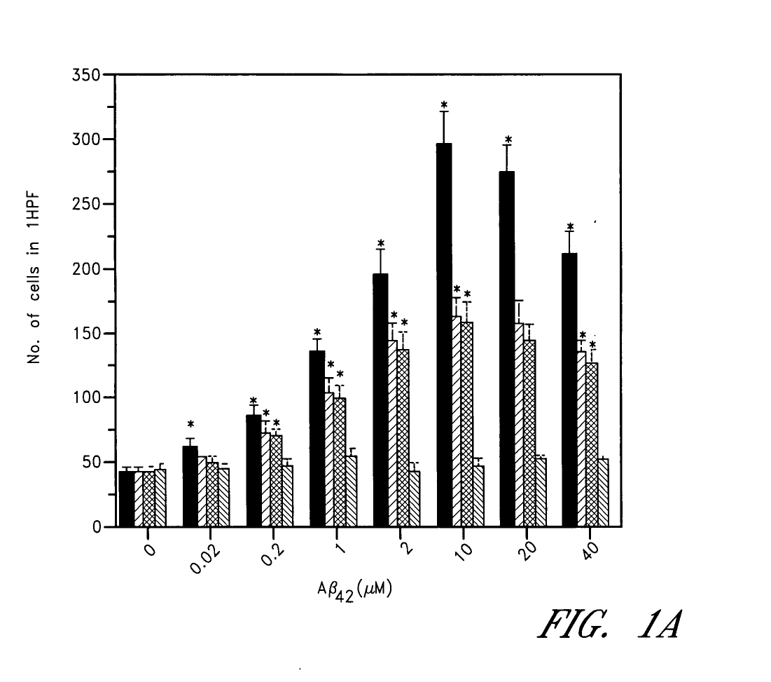

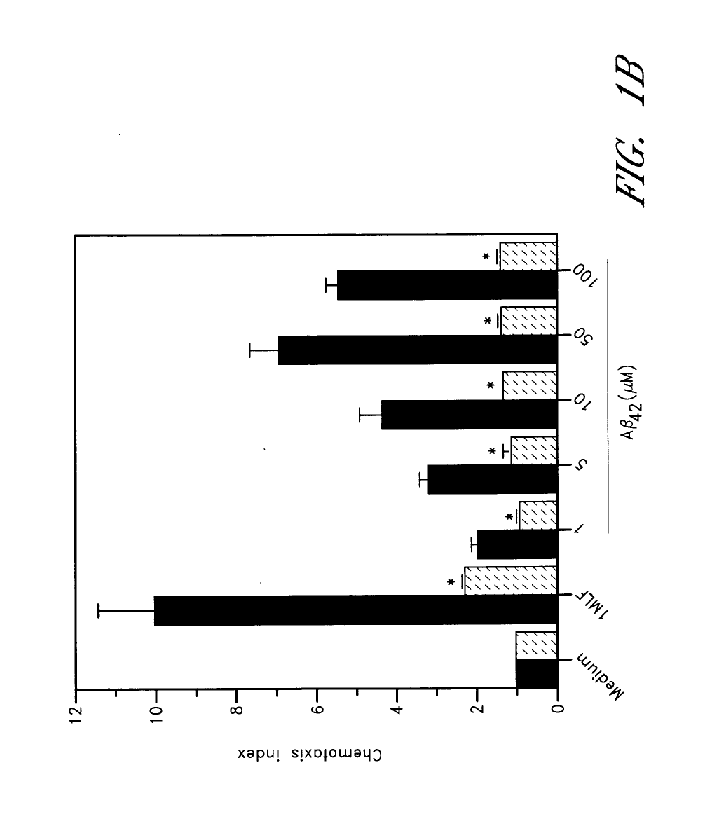

Aβ₄₂ activates monocytes. Microglial cells are considered to belong to the monocyte-macrophage lineage (Neuroinflammatory Working Group 2000 Neurobiol Aging 21:383-421). Extensive studies on the biological activity of Aβ have been performed with human monocytes and monocytic cell lines such as THP-1 with similar activation patterns (Davis J. B. et al. 1992 Biochem Biophys Res Commun 189:1096-1100; London J. A. et al. 1996 PNAS USA 93:4147-4152; Klegeris A. & McGeer P. L. 1997 J Neurosci Res 49:229-235; Klegeris A. et al. 1997 Brain Res 747:114-121; Lorton D. 1997 Mech Ageing Dev 94:199-211; McDonald D. R. et al. 1997 J Neurosci 17:2284-2294; McDonald D. R. et al. 1998 J Neurosci 18:4451-4460; Combs C. K. et al. 1999 J Neurosci 19:928-939). To characterize the nature of the putative receptor or receptors used by Aβ, we studied the effect of Aβ₄₂ on chemotaxis and activation of human monocytes. Freshly dissolved Aβ₄₂ induced a dose-dependent migration of human monocytes starting at a concentration of 20 nM (EC₅₀, 1.5 μM; FIG. 1A). In contrast, peptide with the reverse sequence of Aβ₄₂ (Aβ₄₂₋₁), was completely inactive (FIG. 1A). Checkerboard analysis indicated that Aβ₄₂ functioned chemotactically rather than by increasing random cell migration. Because aggregated Aβ is likely to deposit in senile plaques of AD and activates mononuclear phagocytes in vitro, we tested the chemotactic activity of Aβ₄₂ “aged” at 37° C. FIG. 1A shows that this form of Aβ₄₂ also induced significant monocyte migration, although with lower potency than freshly dissolved peptide. The activation of monocytes by Aβ₄₂ was further demonstrated by increased Ca²⁺ mobilization (FIG. 1C). In both chemotaxis and calcium flux assays, human monocytes responded to a wide range concentrations of Aβ₄₂. These concentrations of Aβ₄₂ are comparable with or much lower than those used in other studies. In addition, preincubation of monocytes with pertussis toxin (PT), an inhibitor of Gᵢ-type proteins, completely abolished monocyte migration (FIG. 1B) and calcium flux in response to Aβ₄₂ (FIG. 1C, inset). These results suggest that Aβ₄₂ uses Gᵢ-protein-coupled STM receptor or receptors on monocytes.

Desensitization of Aβ₄₂ signaling. To identify the monocyte receptor or receptors for Aβ₄₂, we examined the capacity of Aβ₄₂ to cross-desensitize cell signaling with chemoattractants known to elicit Ca²⁺ mobilization. This approach can distinguish between unique and/or shared STM receptors for different chemoattractants (Deng X. et al. 1999 Blood 94:1165-1673). Aβ₄₂ signaling in monocytes was not affected by previous stimulation of the cells with a number of chemokines, suggesting that Aβ₄₂ did not use a chemokine receptor. However, a classical chemoattractant, the bacterial chemotactic peptide formyl-methionyl-leucyl-phenylalanine (fMLF), clearly inhibited the subsequent Ca²⁺ flux response to Aβ₄₂ (FIG. 1D, E). Because high concentrations of fMLF were required, we postulated that Aβ₄₂ might share a low-affinity fMLF receptor. Such a receptor was cloned 10 years ago and has been designated FPRL1 or LXA4R (lipoxin A4 receptor) based on its homology to the high-affinity fMLF receptor FPR (Murphy P. M. 1994 Annu Rev Immunol 12:593-633; Prossnitz E. R. & Ye R. D. 1997 Pharmacol Ther 74:73-102) and its reported function as a lipoxin A4 receptor (Fiore S. et al. 1994 J Exp Med 180:253-260). Moreover, FPRL1 in our previous study has been identified as a functional receptor for serum amyloid A (SAA), which is chemotactic for human leukocytes (Su S. B. et al. 1999 JExp Med 189:395-402) and is one of the major amyloidogenic proteins involved in chronic inflammation in various organs and tissues (Malle E. & De Beer F. C. 1996 Eur J Clin Invest 26:427-435) but has not been implicated in AD.

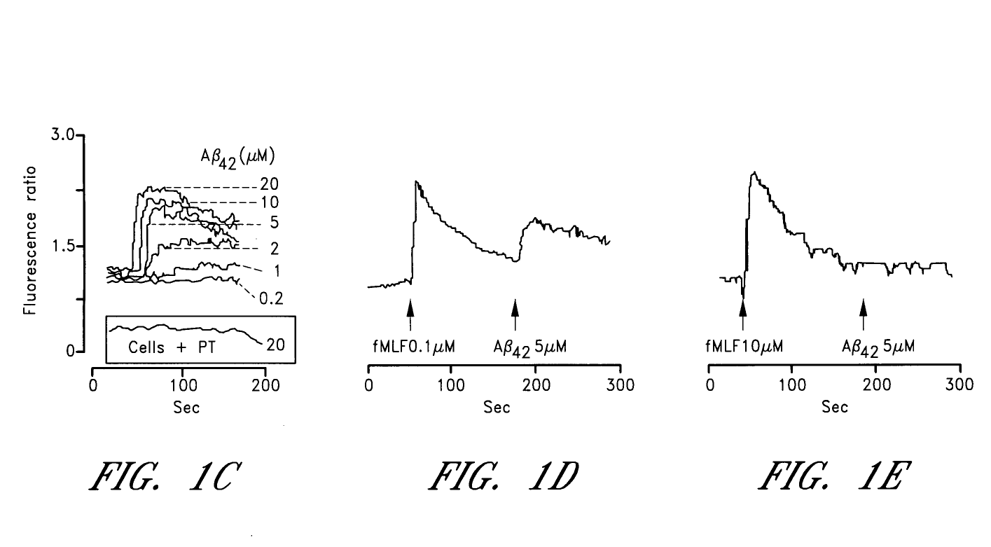

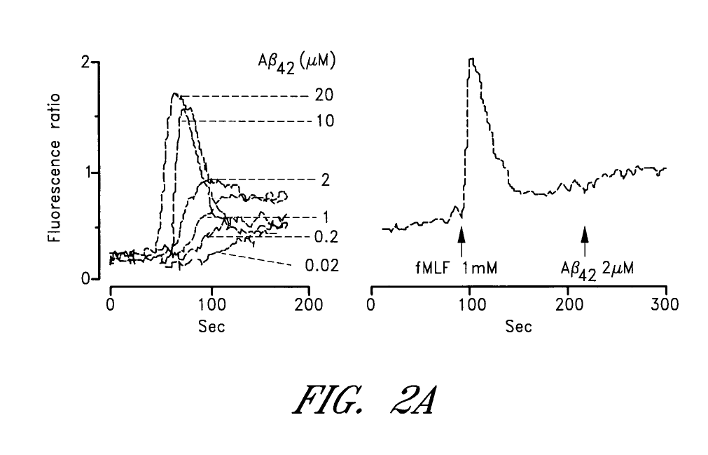

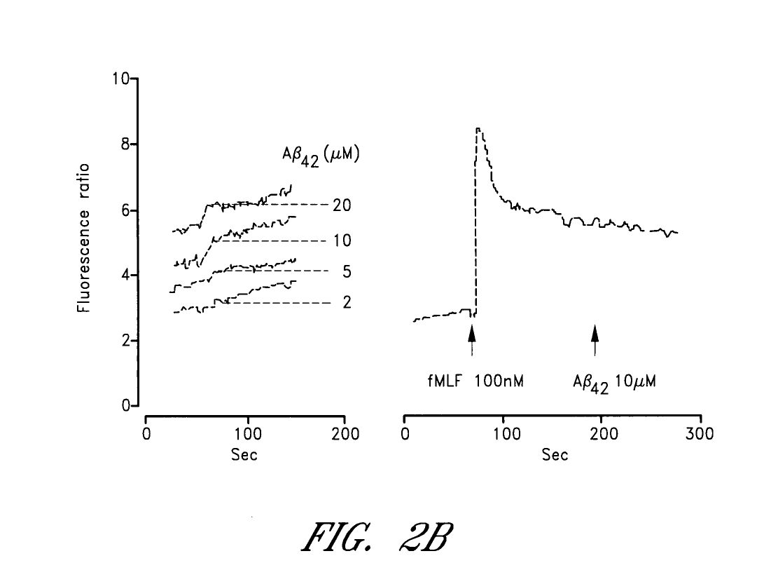

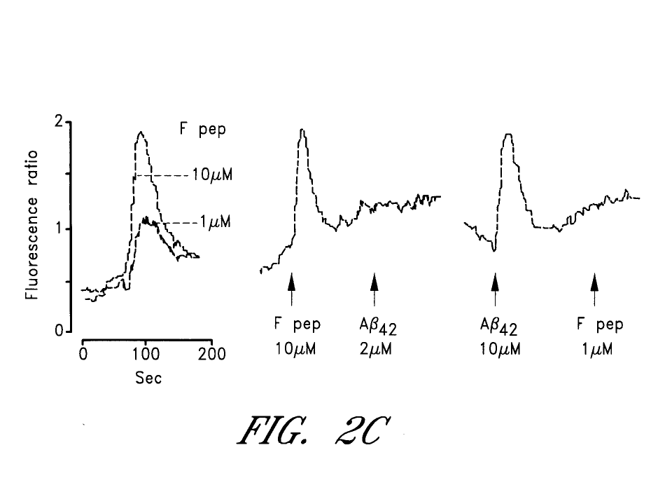

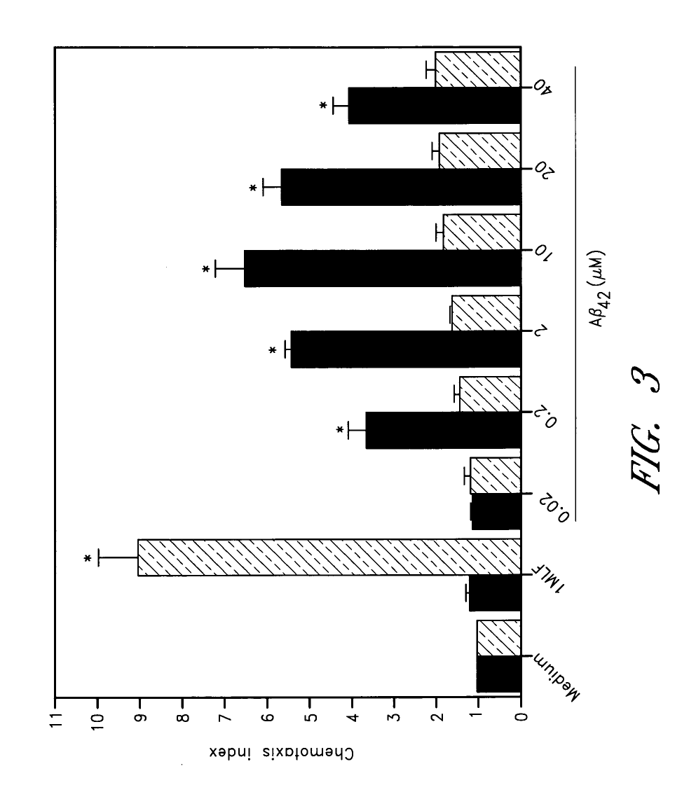

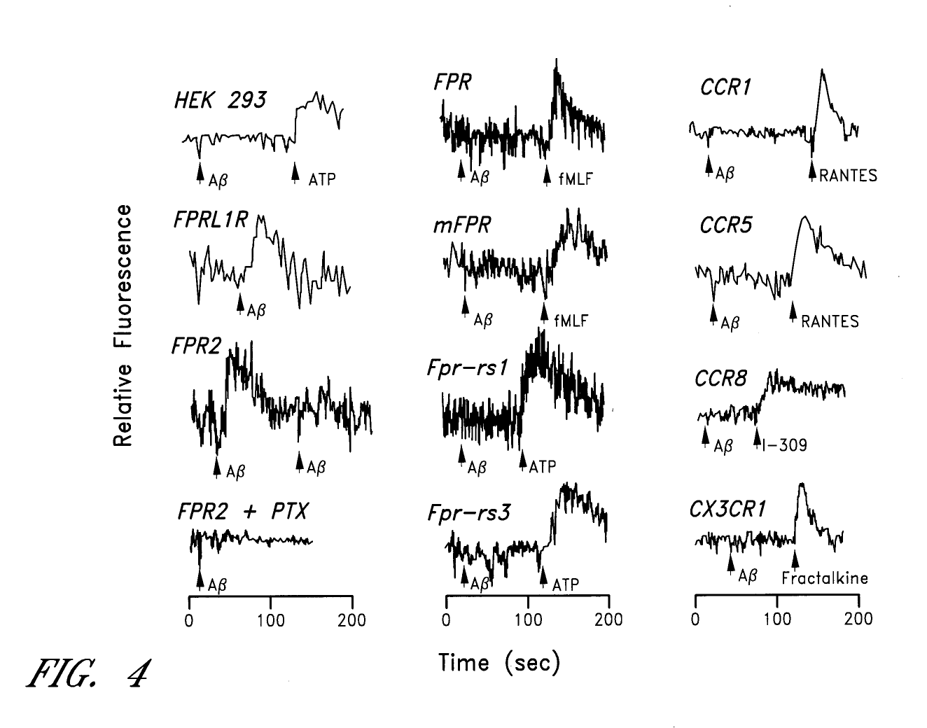

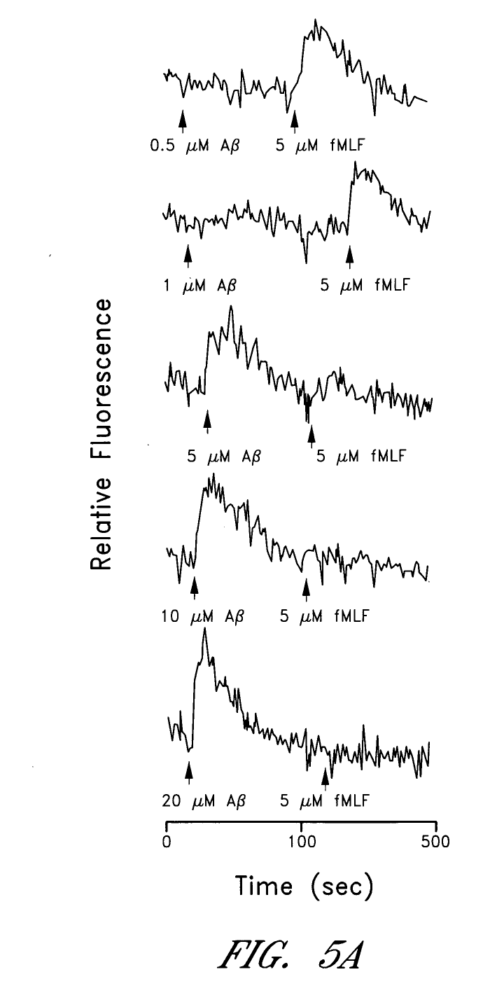

Activation of FPRL1 by Aβ₄₂. We then tested the capacity of Aβ₄₂ to activate cells transfected to express solely FPRL1 or FPR. Aβ₄₂ dose-dependently induced Ca²⁺ mobilization in FPRL1-transfected HEK 293 cells (FPRL1/293 cells) (FIG. 2A). Aβ₄₂ also induced Ca²⁺ mobilization in a rat basophilic leukemia cell line transfected with FPR (ETFR cells), yet with much lower potency and efficacy than fMLF (FIG. 2B). Aβ₄₂ signaling was dependent on FPRL1 and FPR, because untransfected parental cells or cells transfected with other chemoattractant receptors did not respond to Aβ₄₂. Consistent with the effects on monocytes, Aβ₄₂ signaling in both FPRL1/293 and ETFR cells was desensitized by previous stimulation of the cells with high concentrations of fMLF (FIG. 2A, B), which were not toxic to the cells and did not inhibit the cell response to other Ca²⁺ flux inducers. In addition, a synthetic HIV-1 envelope protein domain F peptide, which specifically activates FPRL1(Deng X. et al. 1999 Blood 94:1165-1673), also desensitized Aβ₄₂-induced Ca²⁺ flux in FPRL1/293 cells and vice versa (FIG. 2C). Furthermore, FPRL1/293 cells exhibited a significant chemotactic response to Aβ₄₂ (EC₅₀, 200 nM), whereas ETFR cells migrated only weakly, albeit significantly, in response to high concentrations (>10 μM) of Aβ₄₂ (FIG. 3). The Aβ₄₂ concentrations required to activate FPRL1 is similar to those for monocytes, indicating a major role for FPRL1 in monocyte activation. Because directional cell migration is considered an initial step for cell infiltration and accumulation at sites of inflammation, we propose that FPRL1 is a functionally relevant receptor used by Aβ₄₂.

Expression of FPRL1 gene in AD brain tissue. To gain insight into the pathophysiological relevance of FPRL1 to AD, we examined FPRL1 gene expression in normal versus AD brain tissues. Multiple senile plaques were readily visible with Congo Red staining in sections of brain tissues from AD patients, but not from normal brain. All senile plaques, but not surrounding brain tissue, were infiltrated by cells expressing considerable levels of FPRL1 as determined by in situ hybridization with antisense FPRL1 probe. Hybridization signals were not detected with FPRL1 sense probe in serial sections of senile plaques. The cells infiltrating plaques were positively stained with monoclonal antibody against CD11b, a marker for microglial cells. These results confirm the microglial cell infiltration in AD lesions, and the infiltrating cells express FPRL1.

### Discussion

Aβ peptides have previously been shown to elicit a diverse proinflammatory responses in mononuclear phagocytes, including microglial cells, monocytes, and monocytic cell lines. These include induction of cell adhesion, migration (Davis J B. et al. 1992 Biochem Biophys Res Commun 189:1096-1100; El Khoury J. et al. 1996 Nature 382:716-719; Yan S. D. et al. 1996 Nature 382:685-691; Nakai M. et al. 1998 NeuroReport 9:3467-3470), accumulation at sites of injection in the brain (Scali C. et al. 1999 Brain Res 831:319-321), Ca²⁺ mobilization (Combs C. K. et al. 1999 J Neurosci 19:928-939), phagocytosis (Kopec K. K. & Carroll R. T. 1998 J Neurochem 71:2123-2131), release of reactive oxygen intermediates, and increased production of neurotoxic or proinflammatory cytokines (Bonaiuto C. et al. 1997 J Neuroimmunol 77:51-56; Klegeris A. & McGeer P. L. 1997 J Neurosci Res 49:229-235; McDonald D. R. et al. 1997 J Neurosci 17:2284-2294; Fiala M. et al. 1998 Mol Med 4:480-489). A signal transduction in monocytes involves activation of G-proteins, protein kinase C (Zhang C. et al. 1996 FEBS Lett 386:185-188; Klegeris A. et al. 1997 Brain Res 747:114-121; Lorton D. 1997 Mech Ageing Dev 94:199-211; Nakai M. et al. 1998 NeuroReport 9:3467-3470), and tyrosine kinases (Zhang C. et al. 1996 FEBS Lett 386:185-188; McDonald D. R. et al. 1997 J Neurosci 17:2284-2294; McDonald D. R. et al. 1998 J Neurosci 18:4451-4460; Combs C. K. et al. 1999 J Neurosci 19:928-939), which are known to be activated by STM receptors including FPR and FPRL1 (Murphy P. M. 1994 Annu Rev Immunol 12:593-633; Prossnitz E. R. & Ye R. D. 1997 Pharmacol Ther 74:73-102; Le Y. et al. 1999 Forum (Genova) 9:299-314), but not by the previously reported Aβ receptors SR or RAGE. A recent study reported that the bacterial fMLF and antagonists against the high-affinity fMLF receptor FPR attenuated the production of proinflammatory cytokines induced by Aβ in microglial and THP-1 monocytes, suggesting that Aβ may activate an FPR-like cellular receptor (Lorton D. et al. 2000 Neurobiol Aging 21:463-473). We now have shown that Aβ₄₂ is able to activate FPR, however, the efficacy of this receptor to mediate cell migration and activation is much lower than that of FPRL1. Because Aβ₄₂ induces high levels of chemotaxis and Ca²⁺ flux via FPRL1 on monocytes, and furthermore, the concentrations of Aβ required for cell activation can be detected in AD brain and plasma (Kuo Y. M. et al. 1999 Biochem Biophys Res Commun 257:787-791; McLean C. A. et al. 1999 Ann Neurol 46:860-866), it is evident that in vivo Aβ₄₂ activates mononuclear phagocytes mainly via FPRL1.

### EXAMPLE 2

### Amyloid-β Induces Chemotaxis and Oxidant Stress by Acting at Formylpeptide Receptor 2, a G Protein-Coupled Receptor Expressed in Phagocytes and Brain

Amyloid-β, the pathologic protein in Alzheimer's disease, induces chemotaxis and production of reactive oxygen species in phagocytic cells, but mechanisms have not been fully defined. Here we provide three lines of evidence that the phagocyte G protein-coupled receptor (N-formylpeptide receptor 2 (FPR2)) mediates these amyloid-p-dependent functions in phagocytic cells. First, transfection of FPR2, but not related receptors, including the other known N-formylpeptide receptor FPR, reconstituted amyloid-α-dependent chemotaxis and calcium flux in HEK 293 cells. Second, amyloid-β induced both calcium flux and chemotaxis in mouse neutrophils (which express endogenous FPR2) with similar potency as in FPR2-transfected HEK 293 cells. This activity could be specifically desensitized in both cell types by preincubation with a specific FPR2 agonist, which desensitizes the receptor, or with pertussis toxin, which uncouples it from Gᵢ-dependent signaling. Third, specific and reciprocal desensitization of superoxide production was observed when N-formylpeptides and amyloid-β were used to sequentially stimulate neutrophils from FPR−/− mice, which express FPR2 normally. Biological relevance of these results to the neuroinflammation associated with Alzheimer's disease was indicated by two additional findings: first, FPR2 mRNA could be detected by PCR in mouse brain; second, induction of FPR2 expression correlated with induction of calcium flux and chemotaxis by amyloid-β in the mouse microglial cell line N9. Further, in sequential stimulation experiments with N9 cells, N-formylpeptides and amyloid-β were able to reciprocally cross-desensitize each other. Amyloid-β was also a specific agonist at the human counterpart of FPR2, the FPR-like 1 receptor. These results indicate a unified signaling mechanism for linking amyloid-β to phagocyte chemotaxis and oxidant stress in the brain.

### Introduction

In Alzheimer's disease, progressive dementia and neurodegeneration are associated with a complex pathologic lesion made up of neurofibrillary tangles and aggregated extracellular protein deposits, known as senile plaques, which together are surrounded and infiltrated by activated microglial cells (Selkoe D. J. 1999 Nature 399 (suppl.):A23-A31). Amyloid-β (Aβ), a heterogeneous 39-43-amino acid, self-aggregating peptide produced by sequential cleavage of amyloid precursor protein by the enzymes β-secretase and γ-secretase, is central to the pathogenesis of this disease (Vassar R. et al. 1999 Science 286:735-741; Li Y.-M. et al. 2000 Nature 405:689-694). The main component of senile plaque (Storey E. & Cappai R. 1999 Neuropathol Appl Neurobiol 25:81-97), Aβ is also biologically active and has been proposed to promote neurodegeneration by both direct and indirect mechanisms. It is directly toxic to cultured neurons in vitro (Mattson M. P. 1997 Physiol Rev 77:1081-1132) and is able to regulate production of the protein tau (Lee M. S. et al. 2000 Nature 405:360-364), which accumulates in neurofibrillary tangles. It may also induce neurodegeneration indirectly through its proinflammatory activity (Rogers J. et al. 1996 Neurobiol Aging 17:681-686; London J. A. et al. 1996 PNAS USA 93:4147-4152; Cotter R. L. et al. 1999 J Leukocyte Biol 65:416-427; Meda L. et al. 1995 Nature 374:647-650), which includes the ability to directly induce chemotaxis of mononuclear phagocytes (Davis J. B. et al. 1992 Biochem Biophys Res Commun 189:1096-1100; Fiala M. et al. 1998 Mol Med 4:480-489) as well as production of cytokines and reactive oxygen species (Bianca V. D. et al. 1999 J Biol Chem 274:15493-15499; Lorton D. 1997 Mech Ageing Dev 94:199-211; Araujo D. M. & Cotman, C. W. 1992 Brain Res 569:141-145; Bonaiuto C. et al. 1997 J Neuroimmunol 77:51-56; Sutton E. T. et al. 1999 J Submicrosc Cytol Pathol 31:313-323; McDonald D. R. et al. 1997 J Neurosci 17:2284-2294; El Khoury J. et al. 1996 Nature 382:716-719) by microglial cells, monocytes, and neutrophils. Aβ may also induce phagocyte accumulation and activation indirectly, by inducing C5a production through activation of complement (Bradt B. M. et al. 1998 J Exp Med 188:431-438) or by inducing macrophage colony-stimulating factor release from neurons (Yan S. D. et al. 1997 PNAS USA 94:5296-5301). Consistent with a proinflammatory role, intravascular injection of Aβ causes endothelial cell leakage and leukocyte adhesion and migration in vivo (Sutton E. T. et al. 1999 J Submicrosc Cytol Pathol 31:313-323). The notion that inflammation is important in the pathogenesis of Alzheimer's disease is consistent with clinical reports linking nonsteroidal anti-inflammatory drug administration to reduced incidence of disease and milder clinical course in affected patients (Flynn B. L. & Theesen K. A. 1999 Ann Pharmacother 33:840-849).

The mechanism of Aβ action on cells has not been fully defined yet. Aβ has been reported to bind to several otherwise unrelated receptors, including the receptor for advanced glycation end products (RAGE; Yan S. D. et al. 1996 Nature 382:685-691), the class A scavenger receptor (El Khoury J. et al. 1996 Nature 382:716-719), the p75 neurotrophin receptor (Yaar M. et al. 1997 J Clin Invest 100:2333-2340), glypican (Schulz J. G. et al. 1998 Eur J Neurosci 10:2085-2093), neuronal integrins (Sabo S. et al. 1995 Neurosci Lett 184:25-28), and the N-methyl-D-aspartate receptor (Cowbum R. F. et al. 1997 Neurochem Res 22:1437-1442).

The role of glypican, N-methyl-D-aspartate receptors, integrins, and p75 neurotrophin receptor in mediating Aβ action is not defined. RAGE has been implicated in mediating Aβ-induced oxidant stress in endothelial cells and cortical neurons, NF-κB activation in endothelial cells, and induction of tumor necrosis factor-α production, chemotaxis, and haptotaxis of the mouse microglial cell line BV-2 (El Khoury J. et al. 1996 Nature 382:716-719); conflicting results have been reported with regard to the role of RAGE in Aβ-induced neurotoxicity (Liu Y. et al. 1997 Biochem Biophys Res Commun 237:37-40). Scavenger receptors have been reported to mediate adhesion of rodent microglial cells and human monocytes to Aβ fibril-coated surfaces, leading to secretion of reactive oxygen species and cell immobilization (McDonald D. R. et al. 1997 J Neurosci 17:2284-2294), and to mediate internalization of aggregated Aβ protein (Paresce D. M. et al. 1996 Neuron 17:553-565); however, these receptors do not appear to mediate Aβ stimulation of peripheral blood monocyte-dependent neurotoxicity (Antic A. et al. 2000 Exp Neurol 161:96-101). Aβ has also been reported to have direct toxic effects on membranes independent of receptors (Schubert D. et al. 1995 PNAS USA 92:1989-1993). Despite these advances, the precise mechanisms by which Aβ induces chemotaxis and oxidant production in primary phagocytic cells remain undefined.

Most known phagocyte chemotactic receptors are members of the Gᵢ class of G protein-coupled receptors (GPCRs), which signal through pertussis toxin-sensitive pathways (Murphy P. M. 1994 Annu Rev Immunol 12:593-633). Recently, pertussis toxin was reported to block Aβ induction of interleukin-1 release from the human monocytic cell line THP-1 (Lorton D. 1997 Mech Ageing Dev 94:199-211) as well as Aβ induction of calcium flux in HL-60 cells (Takenouchi T. & Munekata E. 1995 Peptides 16:1019-1024; Correction 1995 Peptides 16:1557). This, together with the fact that calcium flux is strongly associated with G protein-coupled receptor (GPCR) activation by chemoattractants, suggested to us that Aβ may act via a GPCR. Since ligand promiscuity is a common property of chemoattractant receptors, we tested this hypothesis by examining the ability of cloned phagocyte chemoattractant receptors to reconstitute Aβ signaling in a transfected cell line. We also investigated receptors mediating Aβ signaling on mouse phagocytes and human phagocytes.

Cell Lines. Construction of human embryonic kidney (HEK) 293 cell lines expressing human formylpeptide receptor (FPR), human formylpeptide receptor-like 1 receptor (FPRL1R), mouse FPR, mouse FPR2, mouse lipoxin A4 receptor (encoded by Fpr-rs1), a mouse orphan receptor encoded by Fpr-rs3, and human CCR5 and CX3CR1 has been previously described (Hartt J. K. et al. 1999 J Exp Med 190:741-747; Combadiere C. et al. 1996 J Leukocyte Biol 60:147-152; Combadiere C. et al. 1998 J Biol Chem 273:23799-23804; Gao J.-L. et al. 1998 Genomics 51:270-276). Fpr-rs1 and Fpr-rs3 were tested because of their high structural similarity to the known formylpeptide receptors and because they are also expressed in phagocytes (Gao J.-L. et al. 1998 Genomics 51:270-276). Cells were grown in Dulbecco's modified Eagle's medium high glucose medium (Life Technologies, Inc.) containing 10% heat-inactivated fetal calf serum (Hyclone, Logan, Utah), 100 units/ml penicillin, 100 μg/ml streptomycin (Hyclone), and 2 mg/ml G418 (Life Technologies) at 37° C., 5% CO₂, and 100% humidity. A human CCR1-expressing HEK 293 cell line has also been previously described (Combadiere C. et al. 1995 J Biol Chem 270:29671-29675); culture conditions were the same except for usage of 200 units/ml hygromycin B (Calbiochem) as the selective antibiotic. A mouse pre-B cell lymphoma cell line (4DE4) expressing human CCR8 has been reported previously (Tiffany H. L. et al. 1997 J Exp Med 186:165-170). These cells were cultured in RPMI 1640 (Life Technologies) containing 10% heat-inactivated fetal bovine serum, 50 μM β-mercaptoethanol (Sigma), and 2 mg/ml G418. The N9 murine microglial cell line was a kind gift from Dr. P. Ricciardi-Castagnoli (Universita Degli Studi di Milano-Bicocca, Milan, Italy). These cells express typical markers of resting mouse microglia and have been extensively used as representatives of primary mouse microglial cells (Ferrari D. et al. 1996 J Immunol 156:1531-1539). The cells were grown in Iscove's modified Dulbecco's medium supplemented with 5% heat-inactivated fetal calf serum, 2 mM glutamine, 100 units/ml penicillin, 100 μg/ml streptomycin, and 50 mM 2-mercaptoethanol.

Preparation of Mouse Neutrophils. Neutrophils were obtained from the peritoneal cavity of wild type and gene knockout litter mates of F1 and F6 backcrosses of 129/sV FPR−/− mice with C57B1/6 mice 3-4 h after intraperitoneal injection of a 3% thioglycollate solution, as previously described (Gao J-L. et al. 1999 J Exp Med 189:657-662). The cell population was consistently composed of >90% neutrophils, as determined by light microscopy of DiffQuick-stained cytospins. Thus, hereafter we will refer to this cell preparation as neutrophils.

Calcium Flux Analysis. To monitor intracellular Ca²⁺ concentration, adherent cells were harvested by incubation in phosphate-buffered saline at 37° C. for 15 min and then incubated in phosphate-buffered saline containing 2.5 μM Fura-2/AM at 37° C. for 45 min. Cells were washed twice with HBSS (Hanks' balanced salt solution, Life Technologies) and suspended in HBSS at 1-2×10⁶/ml. One ml of cells was added to 1 ml of HBSS and stimulated with ligand in a continuously stirred cuvette at 37° C. in a fluorimeter (model MS-III; Photon Technology Inc., South Brunswick, N.J.). Data were recorded every 200 ms as the relative ratio of fluorescence emitted at 510 nm following sequential excitation at 340 and 380 nm. The following ligands were evaluated: Aβ (nonfibrillated, human residues 1-42; California Peptide Research; Napa, Calif.), fMet-Leu-Phe (fMLF; Sigma), ATP (Life Technologies), and the chemokines RANTES (regulated upon activation normal T-cell expressed and secreted), SDF-1 (stromal cell-derived factor-1), 1-309, fractalkine, MIP-1α (macrophage inflammatory protein-1α), and KC (Peprotech, Rocky Hill, N.J.). The particular chemokines tested were chosen because of their specificity for phagocyte targets. All chemokines were human with the exception of KC, which is mouse. The receptor targets for these chemokines are as follows: RANTES, CCR1, CCR3 and CCR5; SDF-1, CXCR4; I-309, CCR8; fractalkine, CX3CR1; MIP-1α, CCR1, and CCR5; CXCR2. Aβ, chemokines and ATP were dissolved in water and stored at −20° C.; fMLF was dissolved in Me₂SO and stored at −20° C. In some experiments, the cells were incubated in 250 ng/ml pertussis toxin (PTX; Calbiochem) for 4 h at 37° C. in medium, harvested, and loaded with Fura-2/AM as described above. Immediately after harvesting, murine neutrophils were incubated in 1-2×10⁶/ml of phosphate-buffered saline containing 2.5 μM Fura-2/AM for 45 min at 37° C. Neutrophils were washed twice in HBSS and suspended to 1-2×10⁶/ml for analysis. Calcium flux was performed with N9 cells preincubated in the presence or absence of 300 ng/ml lipopolysaccharide (LPS) (37° C., 24 h) using similar procedures.

Chemotaxis. HEK 293 cells were harvested from tissue culture flasks by incubation in trypsin (0.05%)/EDTA (0.1%) (Quality Biologicals, Inc., Gaithersburg, Md.) for 5 min at 37° C. Cells were suspended evenly by vigorous pipetting, and excess Dulbecco's modified Eagle's medium supplemented with 10% fetal bovine serum was then added to block trypsin. Cells were washed twice in Dulbecco's modified Eagle's medium and suspended to a concentration of 4×10⁶ cells/ml in chemotaxis medium (RPMI 1640; 20 mM HEPES (Life Technologies) and 1% bovine serum albumin (ICN Biomedicals Inc., Aurora, Ohio)). Chemoattractants, diluted in chemotaxis medium, were added to the bottom wells of a 96-well chemotaxis plate (Neuro Probe, Inc., Gaithersburg, Md.). A 12-μm pore size membrane was placed on top, and 25 μl of cell suspension containing ˜100,000 cells was placed in the upper chamber. Cells were incubated for 5 h at 37° C., 100% humidity, 5% CO₂. The membrane was carefully removed, and cells in the bottom well were counted using a hemacytometer. Methods for murine neutrophils were the same except that ˜200,000 cells were added to the top of a 5-μm pore size membrane. Chemotaxis assays for N9 cells incubated with or without LPS (300 ng/ml, at 37° C. for 24 h) were performed with 48-well chemotaxis chambers (Neuro Probe). Polycarbonate filters with 8-μm pore size and 90-min incubation at 37° C. were used for measurement of microglial cell migration.

Superoxide Production. Mouse neutrophils were suspended in HBSS containing Ca²⁺ and Mg²⁺ at 10⁶ cells/ml. 50 μl (5×10⁴ cells) were distributed into wells of a 96-well microtiter chemiluminescence plate and incubated at 37° C. for 5 min. Then a mixture of the superoxide-specific chemiluminescence indicator reagent Diogenes (National Diagnostics, Atlanta, Ga.) was added to the cells (50% of total reaction volume) with appropriate stimuli or vehicle control, and superoxide dismutase-inhibitable chemiluminescence was measured in a luminometer (Labsystems Luminoskan; Helsinki, Finland). Data are expressed as integrated luminescence (relative light units) observed during 0.5-s readings obtained at 12-s intervals over a time course of 10 min. For sequential stimulation experiments, 5×10⁴ FPR −/− neutrophils were distributed into microcentrifuge tubes, and test substances were added. The mixture was then immediately transferred to a chemiluminescence plate. After incubation at 37° C. for 8 min (when fMLF was the first stimulus) or 9 min (when Aβ was the first stimulus), Diogenes reagent plus the final stimulus was added, and the activity was monitored for 10 min. To control for desensitization of NADPH oxidase by the first stimulus, cells were stimulated with phorbol 12-myristate 13-acetate (PMA) (100 ng/ml) after the second stimulation, and superoxide was measured for 10 min. To control for scavenging of superoxide by fMLF or Aβ, neutrophils were stimulated simultaneously with (i) PMA (100 ng/ml) and Me₂SO (vehicle for fMLF; 0.2% of the volume in which cells were stimulated); (ii) PMA (100 ng/ml) and fMLF (50 μM); (iii) PMA (100 ng/ml); or (iv) PMA (100 ng/ml) plus Aβ (10 μM). Each condition was tested in triplicate, and the mean of the mean number (grand mean) of superoxide dismutase-inhibitable relative light units throughout the duration of the assay and the corresponding standard errors of grand means were calculated. Differences between conditions were tested for significance by two-tailed paired t tests or unequal variance tests (Mann-Whitney rank sum) where appropriate. A value of p<0.05 indicated significant differences.

RNA Analysis by PCR. Wild type littermates of an F7 backcross of 129/sV FPR−/− mice with C57B1/6 mice were euthanized by cervical dislocation, and brains from three mice were removed, pooled, and washed in phosphate-buffered saline for 15 min at room temperature. Brain tissue was sliced in a Petri dish on ice using a clean razor blade and homogenized in an ice-cold Teflon homogenizer. RNA was extracted using the RNA STAT-60 kit (Tel-Test, Inc., Friendswood, Tex.) according to the manufacturer's instructions. RNA was reverse-transcribed using the cDNA Cycle Kit (Invitrogen, San Diego, Calif.) following the manufacturer's instructions. Gene-specific primers were used for PCR amplification of the cDNA using the GeneAmp PCR System 9700 (PerkinElmer Life Sciences). For mouse FPR2, the 5′ primer 5′-TCTACCATCTCCAGAGTTCTGTGG (SEQ ID NO: 2) and 3′ primer 5′-TTACATCTACCACAATGTGAACTA (SEQ ID NO: 3) were used to generate a 268-base pair product. The PCR conditions for amplification were 3 min at 95° C. for the initial melting followed by 30 cycles of 1 min of melting at 95° C., 1 min of annealing at 55° C., 2 min of synthesis at 72° C., with a final extension of 10 min at 72° C. and cooling to 4° C. PCR products were analyzed by gel electrophoresis using a 1% agarose gel in TBE containing 10 μg of ethidium bromide/100 ml. Data were recorded on a UVP Gel Imaging System (Appropriate Technical Resource, Laurel, Md.). For analysis of the N9 microglial cell line, RT-PCR was performed with 0.5 μg of total RNA extracted from cells treated with 300 ng/ml LPS for different time periods (High Fidelity ProSTARTM HF System, Stratagene, Kingsport, Tenn.). The procedure consisted of a 15-min reverse transcription at 37° C., 1-min inactivation of Moloney murine leukemia virus reverse transcriptase at 95° C., and 40 cycles of denaturing at 95 C (30 s), annealing at 55° C. (30 s), and extension at 72° C. (1 min), with a final extension for 10 min at 72° C. Primers for murine-actin gene were used as controls (Stratagene). The RT-PCR products at different dilutions were electrophoresed on 1% agarose gel and visualized with ethidium bromide staining.

Mouse FPR2 and its human counterpart FPRL1R are receptors for amyloid β. Using induction of calcium flux as a highly sensitive and specific real time assay of receptor activation, we screened a panel of stable cell lines transfected with plasmids encoding the known phagocyte formylpeptide receptors (human and mouse FPR, human FPR-like 1 receptor (FPRL1R), and mouse FPR2), four chemokine receptors (human CCR1, CCR5, CCR8, and CX3CR1), the mouse lipoxin A4 receptor (encoded by mouse Fpr-rs1), and an orphan receptor highly related in sequence to formylpeptide receptors (Fpr-rs3), as well as untransfected control cells, for responsiveness to 10 μM Aβ (FIG. 4). This concentration was chosen based on Aβ dose-response studies published previously for human neutrophils and monocytes and rat microglial cells (Bianca V. D. et al. 1999 J Biol Chem 274:15493-15499). The lipoxin A4 receptor and Fpr-rs3 were included because of their high sequence similarity to the formylpeptide receptors (Gao J.-L. et al. 1998 Genomics 51:270-276).

Aβ induced a response in HEK 293 cells expressing FPRL1R and FPR2, which are human and mouse low affinity formylpeptide receptors, respectively. Activation of each receptor produced a robust transient that was similar in magnitude and duration to the response induced by the prototypical N-formylpeptide fMLF in the same cells (FIG. 5, A and B) and was similar kinetically to the transients induced by other classic chemoattractants and chemokines (FIG. 4). Aβ was specific for these receptors, since none of the other cell lines tested responded. The CCR1, CCR5, CCR8, and CX3CR1 and the human and mouse FPR (high affinity formylpeptide receptor) cell lines did respond to appropriate known agonists as previously described (Hartt J. K. et al. 1999 J Exp Med 190:741-747; Combadiere C. et al. 1996 J Leukocyte Biol 60:147-152; Combadiere C. et al. 1998 J Biol Chem 273:23799-23804). The Fpr-rs1 and Fpr-rs3 cell lines were unresponsive to fMLF but did respond to ATP through an endogenous signaling pathway. Although RNA for Fpr-rs1 and Fpr-rs3 is present in these two cell lines, we have not yet obtained direct evidence of receptor protein expression.

Aβ signaling could be completely blocked by pretreatment of the cells with pertussis toxin (FIG. 4, column 1, tracing labeled FPR2+PTX), which inactivates Gᵢ type G proteins. Pertussis toxin also blocks signaling by other FPR2 agonists (Hartt J. K. et al. 1999 J Exp Med 190:741-747; Liang T. S. et al. 2000 Biochem Biophys Res Commun 270:331-335; Hartt J. K. et al. 2000 Biochem Biophys Res Commun 272:699-704). When FPR2 and FPRL1R-expressing cells were sequentially stimulated with 10 μM Aβ, they responded to the first but not the second stimulus (FIG. 4, column 1, tracing labeled FPR2) indicating homologous desensitization of the signal transduction pathway, which is characteristic of G protein-coupled receptors (Ali H. et al. 1999 J Biol Chem 274:6027-6030). Moreover, Aβ and fMLF reciprocally interfered with each other's signaling at FPR2 (FIG. 5, A and B) in a concentration-dependent manner, providing further evidence that both agonists act at the same receptor. This was specific, since Aβ did not affect signaling by agonists acting at any of the other receptors considered (FIG. 4).

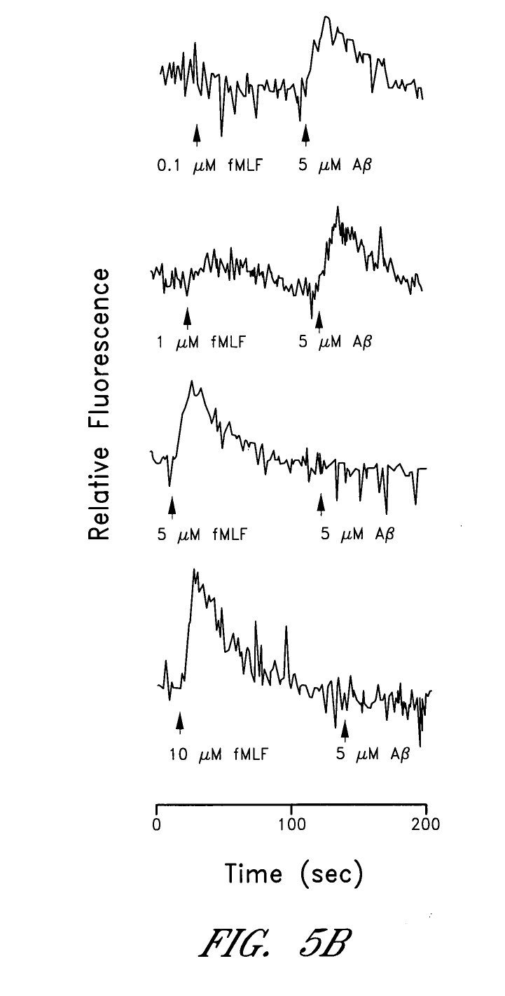

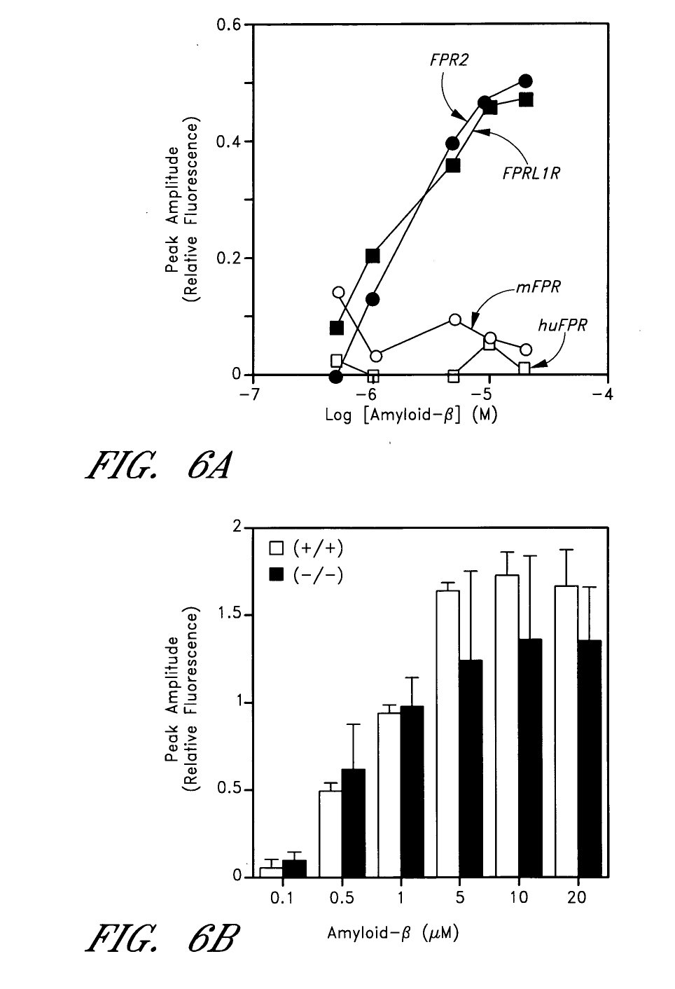

Aβ induced calcium flux in both FPR2- and FPRL1R-transfected HEK 293 cells in a graded concentration-dependent manner, with an EC₅₀ of 5 μM (FIG. 6A). In contrast, HEK 293 cells expressing either mouse or human FPR did not respond to Aβ from 0.5 to 20 μM (FIG. 6A). However, all four cell lines responded to fMLF in a concentration-dependent manner, with EC₅₀ consistent with those previously reported (Hartt J. K. et al. 1999 J Exp Med 190:741-747).

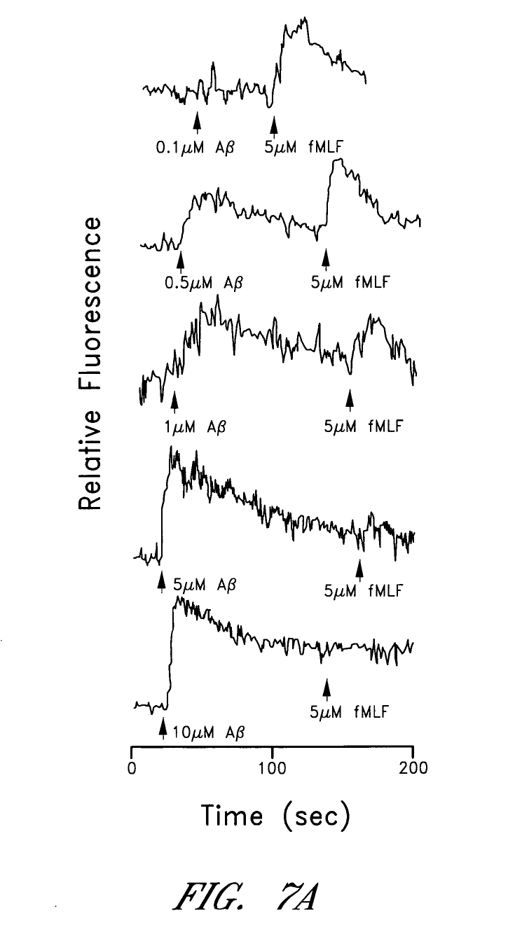

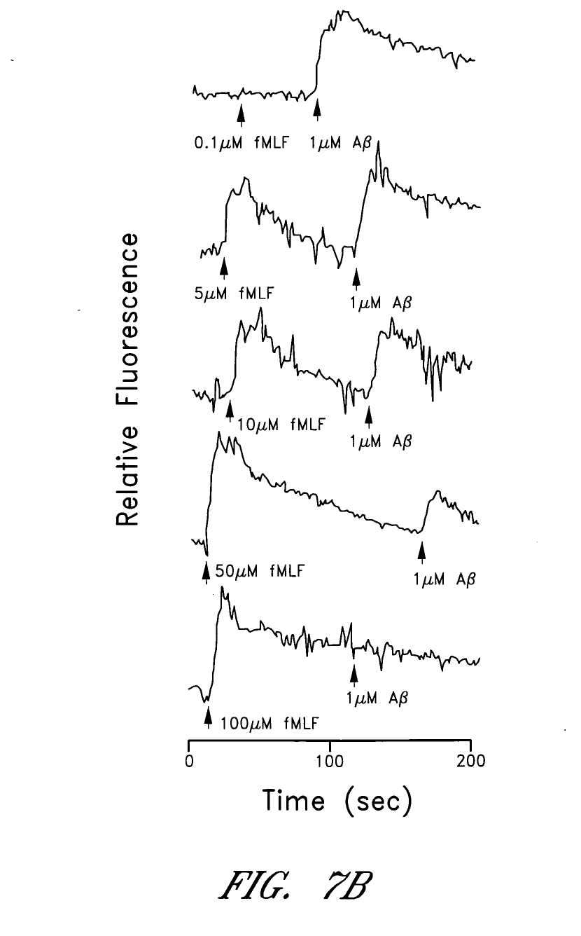

To test whether native FPR2 also functions as an Aβ receptor, we first focused on primary mouse neutrophils, which, as we have previously shown, express FPR2 endogenously (Hartt J. K. et al. 1999 J Exp Med 190: 741-747) and which can be analyzed in an FPR-deficient background due to the availability of FPR knockout mice (Gao J-L. et al. 1999 J Exp Med 189:657-662). Aβ induced calcium flux in FPR −/− neutrophils with an EC₅₀ of 1 μM, similar to the value for FPR2-transfected HEK 293 cells (FIG. 6, A and B). FPR −/− neutrophils also mimicked FPR2-transfected HEK 293 cells in sequential stimulation experiments; fMLF and Aβ were able to reciprocally cross-desensitize each other (FIG. 7, A and B). Specificity was again confirmed by the lack of cross-desensitization in this assay between Aβ and either SDF-1, MIP-1α, or KC in mouse neutrophils (FIG. 7C). It is important to note that FPR and FPR2 both mediate fMLF signaling in mouse neutrophils (Hartt J. K. et al. 1999 J Exp Med 190:741-747; Gao J-L. et al. 1999 J Exp Med 189: 657-662). However, the desensitization experiments were carried out using neutrophils from FPR knockout mice, which rules out cross-desensitization of Aβ action by fMLF signaling through FPR and strongly implicates Aβ usage of endogenous neutrophil FPR2, the only other known neutrophil fMLF receptor. As with FPR2-transfected HEK 293 cells, Aβ induction of calcium flux in mouse neutrophils was completely blocked by pretreatment of the cells with pertussis toxin, indicating a Gᵢ-dependent signaling pathway (FIG. 7C). Aβ potency was indistinguishable in neutrophils from FPR −/− and +/+ mice (FIG. 6B). Although there was a trend toward lower efficacy (maximal response) in cells from FPR −/− mice, this difference was not statistically significant (FIG. 6B).

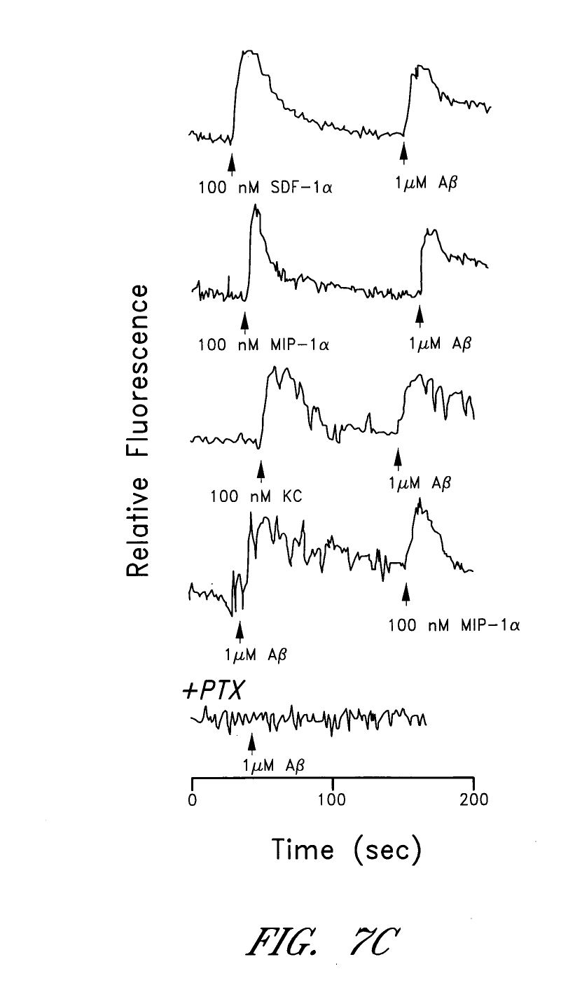

Aβ is a chemotactic agonist at FPR2. To assess the potential biological significance of Aβ-FPR2 signaling, we used in vitro chemotaxis assays as a model of cell migration. Consistent with the calcium flux results, Aβ induced chemotaxis of FPR2-transfected HEK 293 cells but not mouse FPR-transfected HEK 293 cells; likewise, Aβ induced migration of mouse neutrophils (FIG. 8). In each case, the peak responses occurred at −10 μM, and the EC₅₀ values were consistent with the values for induction of calcium flux in these cells, 5 μM (FIG. 8, B and C). We have previously shown that the fMLF dose-response curve for chemotaxis in neutrophils from wild type mice has two peaks, one with an optimum at ˜500 nM and the other with an optimum at 10 μM. The 500 nM optimum is due to FPR activity, since it is absent in cells from FPR −/− mice (Hartt J. K. et al. 1999 J Exp Med 190:741-747). The second peak is consistent with FPR2 pharmacology in transfected HEK 293 cells. Since the dose-response curve for Aβ chemotaxis is the same in neutrophils from FPR −/− and +/+ mice, Aβ chemoattraction of mouse neutrophils is not mediated by FPR. Since in FPR −/− neutrophils the Aβ and fMLF chemotactic and calcium flux optima are similar and match the Aβ optimum in FPR2-transfected HEK 293 cells, Aβ chemoattraction of these cells is most likely mediated by FPR2. Since application of Aβ on both sides of the chemotaxis filter gave net results equivalent to the background control, we conclude that Aβ-induced cell migration was due to chemotaxis, not chemokinesis (FIG. 8A).

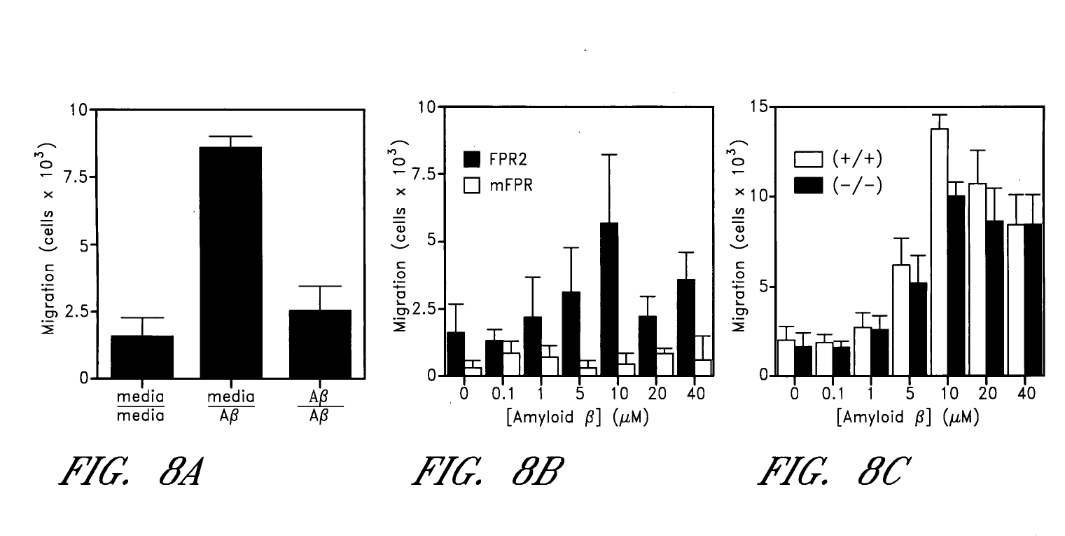

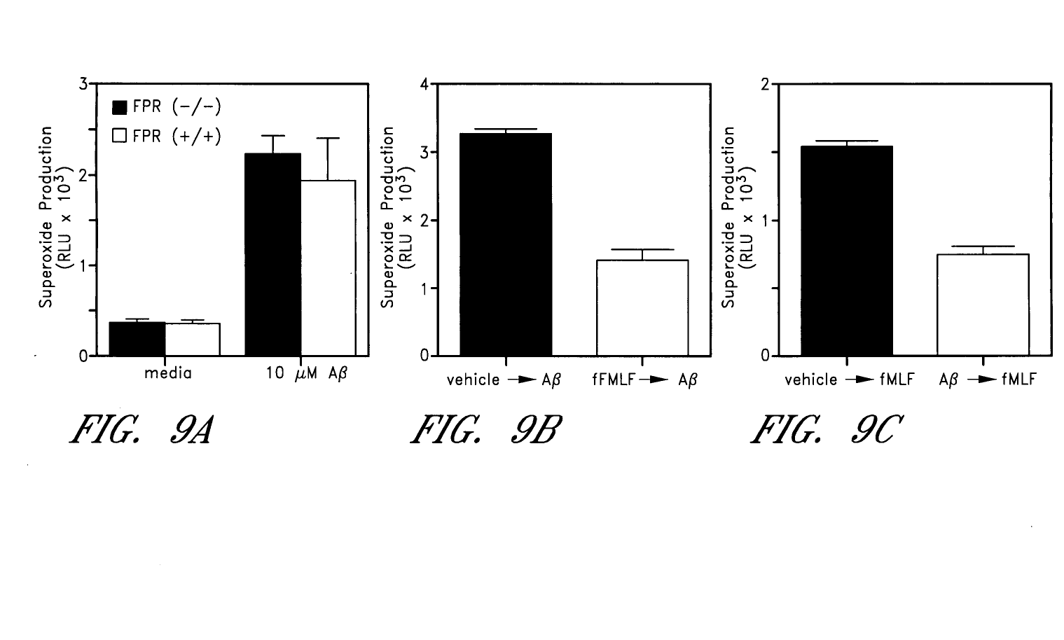

Evidence that FPR2 mediates induction of superoxide generation by amyloid. To test whether FPR2 can also mediate production of reactive oxygen species by Aβ, we examined whether Aβ could induce superoxide production in mouse neutrophils and, if so, whether this activity could be desensitized by prestimulation with fMLF. Again, FPR −/− neutrophils were used to eliminate the possibility of cross-desensitization of AD activity by fMLF signaling through FPR. As shown in FIG. 9A, Aβ at 10 μM, a concentration that saturated the chemotactic and calcium flux response in mouse neutrophils and FPR2/HEK 293 cell transfectants, induced superoxide production with similar efficacy in FPR −/− and FPR+/+ neutrophils. This is consistent with the calcium flux and chemotaxis results. Additional experiments (n=2) showed a similar graded Aβ dose-response relationship and equivalent potency for FPR −/− versus FPR+/+ neutrophils. This is consistent with the chemotaxis and calcium flux results and indicates that Aβ induction of superoxide generation is not mediated by FPR. fMLF also induced superoxide generation in both FPR+/+ and −/− neutrophils; however, the EC₅₀ was 10-fold lower at FPR −/− neutrophils, which is consistent with our previous report of weaker potency of fMLF at FPR2 versus FPR for induction of both calcium flux and chemotaxis in both neutrophils and receptor-transfected cells (Hartt J. K. et al. 1999 J Exp Med 190:741-747).

The superoxide response of FPR −/− neutrophils to 10 μM Aβ was markedly attenuated when the cells were pretreated with 5 μM fMLF compared with pretreatment with vehicle alone (FIG. 9B). Likewise, the response to 5 μM fMLF was markedly attenuated when the cells were pretreated with 10 μM Aβ (FIG. 9C). The reduced response is not due to depletion or inactivation of NADPH oxidase by the first stimulation, because PMA could induce large amounts of superoxide production in cells when added after completion of the response to the second stimulus. Moreover, costimulation experiments in which PMA was added simultaneously with fMLF or Aβ ruled out scavenging as the mechanism by which each agent reduced superoxide production by the other.

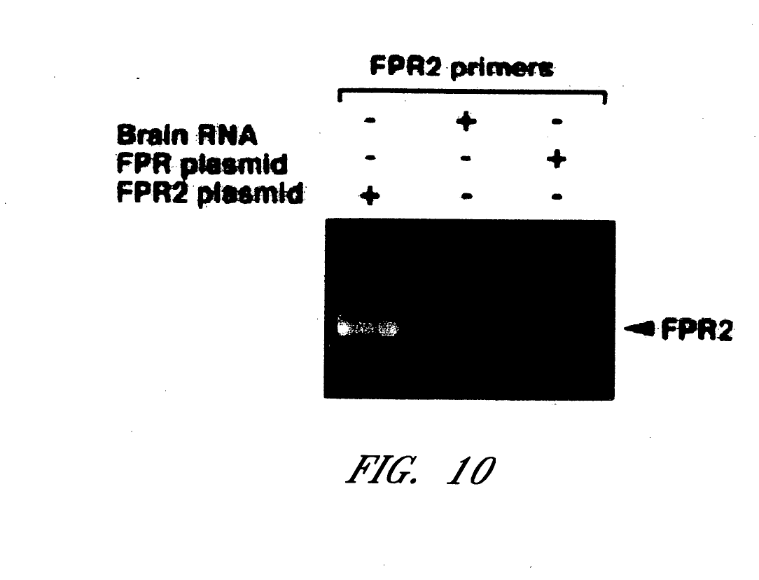

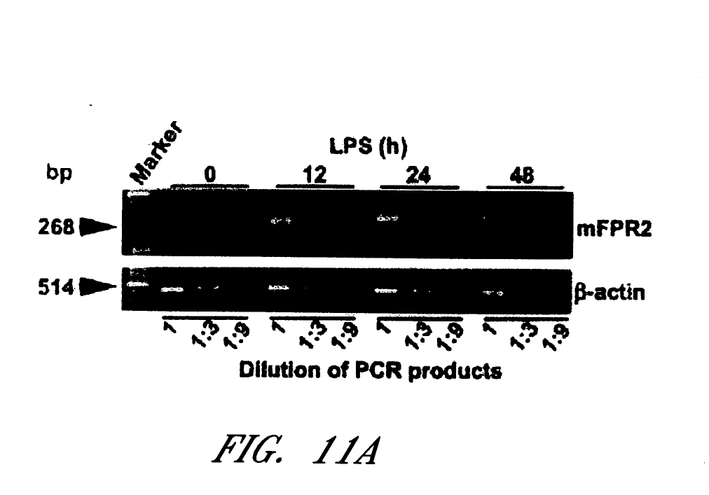

FPR2 is expressed in mouse brain. Previously, we reported that by Northern blot analysis FPR2 mRNA was detectable in mouse spleen, lung, and liver but not brain (Hartt J. K. et al. 1999 J Exp Med 190:741-747). Because of the importance of Aβ to the pathogenesis of Alzheimer's disease and our finding that it is an agonist at FPR2, we reexamined brain expression of FPR2 by RT-PCR (FIG. 10) and were able to detect a relatively weak band of the appropriate size, 268 base pairs.

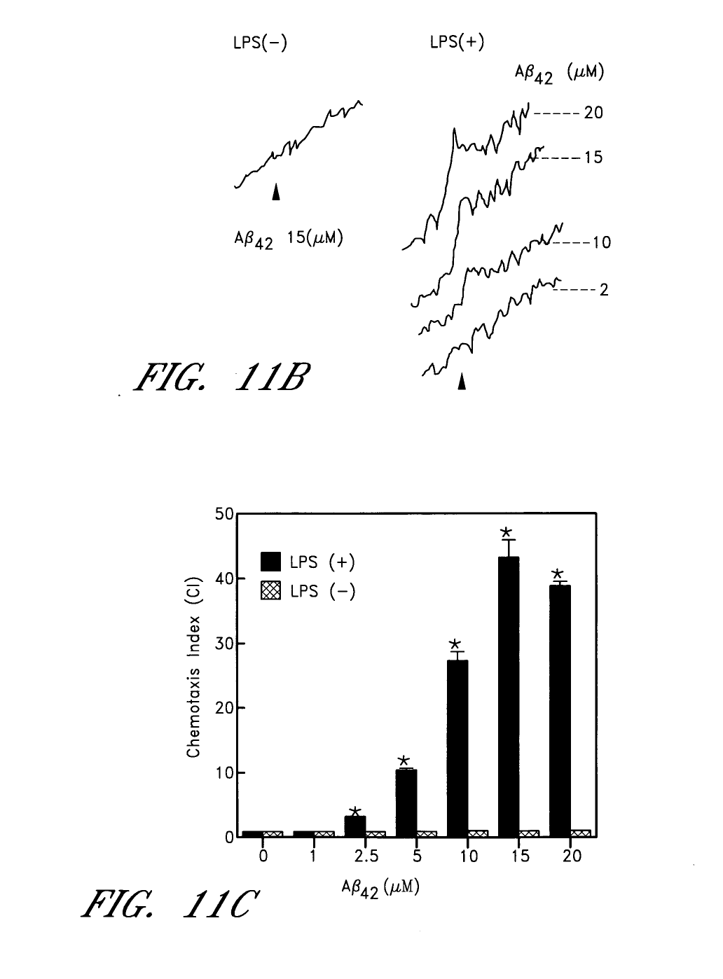

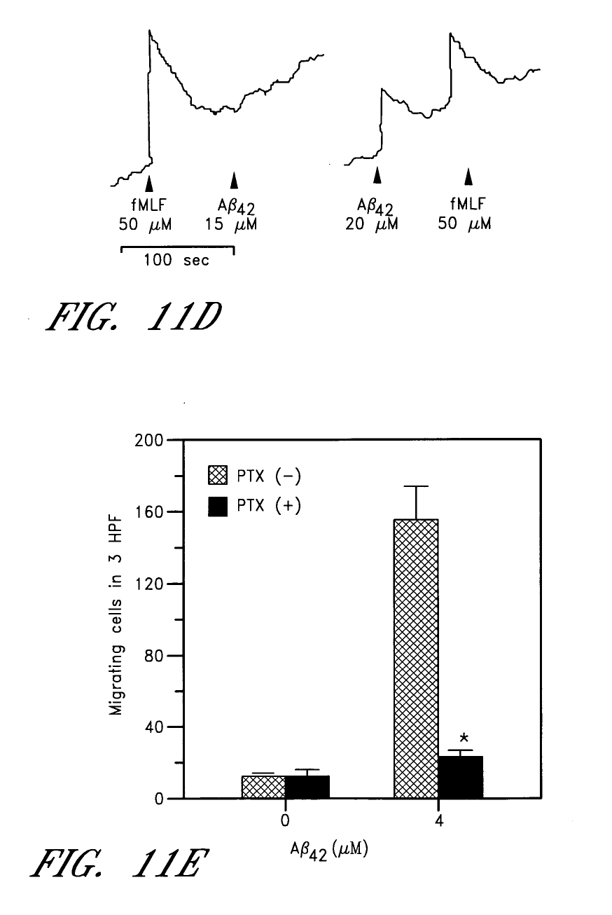

FPR2 expression in a mouse microglial cell line. We next tested whether microglial cells, the major phagocytic cells of the central nervous system, expressed FPR2. For this purpose, we used the murine microglial cell line N9, which expresses typical markers of resting mouse microglia and has been extensively used as a representative of primary mouse microglial cells (Ferrari D. et al. 1996 J Immunol 156:1531-1539). Low levels of FPR2 mRNA could be detected in this cell line under resting conditions using RT-PCR; however, the cells did not respond to Aβ either in calcium flux or chemotaxis assays (FIG. 11). Cell activation with LPS induced FPR2 mRNA expression in a time-dependent fashion (FIG. 8A) and rendered the cells responsive to Aβ in a concentration-dependent manner in both calcium flux and chemotaxis assays (FIG. 11, B and C). The potency of Aβ was consistent for both functions and was consistent with the values obtained in studies of mouse neutrophils and FPR2HEK 293 cells. As we observed with mouse neutrophils and FPR2-transfected HEK 293 cells, fMLF and Aβ were able to reciprocally cross-desensitize each other in sequential stimulation experiments using calcium flux as the functional readout (FIG. 11D). Finally, chemotaxis of LPS-activated N9 cells to Aβ was completely blocked by pretreatment of the cells with pertussis toxin (FIG. 11E), demonstrating a Gᵢ-dependent signaling pathway. This is consistent with the results obtained using the calcium flux assay in neutrophils and FPR2-transfected HEK 293 cells (FIGS. 4 and 7).

### EXAMPLE 3

### βAmyloid Peptide (Aβ₄₂) is Internalized Via the G-Protein-Coupled Receptor FPRL1 and Forms Fibrillar Aggregates in Macrophages

The 42 amino acid form of β amyloid (Aβ₄₂) plays a pivotal role in neurotoxicity and the activation of mononuclear phagocytes in Alzheimer's disease (AD). Our study revealed that FPRL1, a G-protein-coupled receptor, mediates the chemotactic and activating effect of Aβ₄₂ on mononuclear phagocytes (monocytes and microglia), indicating that FPRL1 is involved in the proinflammatory responses in AD. We investigated the role of FPRL1 in cellular uptake and the subsequent fibrillar formation of Aβ₄₂ by using fluorescence confocal microscopy. We found that upon incubation with macrophages or HEK293 cells genetically engineered to express FPRL1, Aβ₄₂ associated with FPRL1 and the Aβ₄₂/FPRL1 complexes were rapidly internalized into the cytoplasmic compartment. The maximal internalization of Aβ₄₂/FPRL1 complexes occurred by 30 min after incubation. Removal of free Aβ₄₂ from culture supernatants at 30 min resulted in a progressive recycling of FPRL1 to the cell surface and degradation of the internalized Aβ₄₂. However, persistent exposure of the cells to Aβ₄₂ over 24 h resulted in retention of Aβ₄₂/FPRL1 complexes in the cytoplasmic compartment and the formation of Congo red positive fibrils in macrophages but not in human embryonic kidney (HEK) 293 cell transfected with FPRL1. These results indicate that besides mediating the proinflammatory activity of Ap₄₂, FPRL1 is also involved in the internalization of Aβ₄₂, which culminates in the formation of fibrils only in macrophages.

### Introduction

Alzheimer's disease (AD) is a progressive neurodegenerative disease characterized by the presence of senile plaques in the brain tissue (Selkoe D. J. 1999 Nature 399:(Suppl.) A23-A31). Although the precise mechanisms of pathogenesis of AD remain undefined, it is well established that the 42 amino acid form of the β amyloid peptide (Aβ₄₂) plays a central role in mediating neurotoxicity and the formation of senile plaques. Elevated level of Aβ₄₂, both in nonfibrillar (Hartley D. M. et al. 1999 J Neurosci 19:8876-8884; Lambert M. P. et al. 1998 PNAS USA 95:6448-6645) and fibrillar (Lorenzo A. & Yankner B. A. 1994 PNAS USA 91:12243-12247) forms, can be directly cytotoxic to neuronal cells; soluble nonfibrillar Aβ₄₂ in particular has been implicated for neuronal loss at the early stages of AD (reviewed in: Klein W. L. et al. 2001 Trends Neurosci 24:219-224). Aβ₄₂ may activate mononuclear phagocytes in the brain and elicit inflammatory responses (Pachter J. S. 1997 Mol Psychiatry 2:91-95; McGeer P. L. & McGeer E. G. 1999 J Leukoc Biol 65:409-415; Kalaria R. N. 1999 Curr Opin Hematol 6:15-24; Neuroinflammatory Working Group 2000 Neurobiol Aging 21:383-342). In fact, previous studies also suggest that the neurotoxicity of Aβ₄₂ may depend on the presence of mononuclear phagocytes (London J. A. et al. 1996 PNAS USA 93:4147-4152). There are two types of mononuclear phagocytes in the brain: perivascular macrophages and microglia; these are thought to be derived from circulating monocytic precursor cells that infiltrate the central nervous system during development as well as at various times postnatally (Hickey W. F. & Kimura H. 1998 Science 239:290-292; Ling E. A. & Wong W. C. 1993 Glia 7:9-18). Histological studies revealing activated microglia and perivascular macrophages closely associated with the dense cores in AD tissue support the hypothesis that these cells are actively involved in this disease. In vitro, Aβ peptides are taken up by monocytes and microglia. They stimulate these cells to release proinflammatory cytokines and neurotoxic mediators (Colton C. A. & Gilbert D. L. 1987 FEBS Lett 223:284-288; Chao C. C. et al. 1992 J Immunol 149:2736-2741; van der Laan L. J. et al. 1996 J Neuroimmunol 70:145-152). This may account for the observations that anti-inflammatory drugs delay the onset of the AD dementia (Pachter J. S. 1997 Mol Psychiatry 2:91-95; McGeer P. L. & McGeer E. G. 1999 J Leukoc Biol 65:409-415; Kalaria R. N. 1999 Curr Opin Hematol 6:15-24; Neuroinflammatory Working Group 2000 Neurobiol Aging 21:383-342; McGeer P. L. et al. 1996 Neurology 47:425-432; Hull M. et al. 1999 Drug Discov Today 4:275-282), supporting the hypothesis that the pathogenesis and progress of AD involve a proinflammatory process in the brain.

Aβ₄₂ activates human mononuclear phagocytes typically through a receptor-mediated signaling pathway, prompting the search for cell surface receptors for Aβ₄₂. Scavenger receptor (SR) and receptor for advanced glycation end products (RAGE) have been proposed as putative receptors of Aβ₄₂ (El Khoury J. et al. 1996 Nature 382:716-719; El Khoury J. et al. 1998 Neurobiol Aging 19:S81-S84; Paresce D. M. et al. 1996 Neuron 17:553-565; Yan S. D. et al. 1996 Nature 382:685-691). However, some studies failed to confirm the capacity of SR or RAGE to mediate the proinflammatory activity of Aβ₄₂ in mononuclear phagocytes. The presence of alternative Aβ₄₂ receptors on such cells has been postulated (Antic A. et al. 2000 Exp Neurol 161:96-101; McDonald D. R. et al. 1998 J Neurosci 18:4451-4460; Lorton, D. et al. 2000 Neurobiol Aging 21:463-473; Combs, C. K. et al. 1999 J Neurosci 19:928-939). We found that a G-protein-coupled, seven-transmembrane receptor, FPRL1, mediates the migration and activation of monocytes and microglia induced by Aβ₄₂ (see Examples 1 and 2). Cells highly expressing the FPRL1 gene were detected in and around the senile plaques in the brain tissues of AD patients (Paresce D. M. et al. 1996 Neuron 17:553-565). These cells were also stained positively for CD11b, a marker typical for mononuclear phagocytes in the brain. Thus, FPRL1 is envisioned as being a relevant cell surface receptor that accounts for the inflammatory responses elicited by Aβ₄₂. The present study aimed to define the effect of FPRL1 on Aβ₄₂ uptake by human mononuclear phagocytes. We report that after binding to FPRL1, Aβ₄₂ is rapidly internalized into the cytoplasmic compartment of the cells; with time, the internalized Aβ₄₂ forms fibrils only in macrophages.

Reagents and cells. W peptide (WKYMVm, W pep), a potent agonist for FPRL1 (Le Y. et al. 1999 J Immunol 163:6777-6784), was custom-synthesized by the Department of Biochemistry, Colorado State University (Fort Collins, Colo.). Aβ₄₂ peptide (Aβ₄₂) was purchased from California Peptide Research (Napa, Calif.). Mouse monoclonal anti-human amyloid β antibody was purchased from Sigma (St. Louis, Mo.). A rabbit polyclonal anti-FPRL1 antiserum was generated against a synthetic peptide derived from the carboxyl-terminal 20 amino acids of human FPRL1 conjugated to Keyhole limpet hemocyanin. IgG was purified from the anti-FPRL1 serum by using Mab Trap G 2 kit from Amersham Pharmacia Biotech, Inc. (Piscataway, N.J.). The purified antibody recognizes FPRL1 in macrophages and in HEK293 cells transfected with this receptor, but does not stain parental HEK293 cells or cells transfected with chemokine receptors. The preimmune serum does not react with FPRL1.

Human peripheral blood monocytes (PBM) were isolated from Buffy coats (Transfusion Medicine Department, NIH Clinical Center, Bethesda, Md.) by using iso-osmotic Percoll gradient. The purity of cell preparations by morphology was >90%. PBM were further differentiated to macrophages by culturing the cells in RPMI 1640 medium containing 0.1% bovine serum albumin, 0.01M HEPES (pH 7.4), and 20 ng/ml monocyte colony stimulating factor (MCSF, Pepro Tech, Rocky Hill, N.J.). The cells were plated on 4-well chamber slides (Nalge Nunc International, Rochester, N.Y.) at a density of 1×10⁵ cells/well. HEK293 cells genetically engineered to express FPRL1 cDNA (FPRL1/293 cells) were kindly provided by Dr. P. M. Murphy (National Institute of Allergy and Infectious Diseases, NIH). FPRL1/293 cells were suspended in DMEM supplemented with 10% FBS (Hyclone, Logan, Utah), 1 mM glutamine (Gibco-BRL, Grand Island, N.Y.), and 800 μg/ml geneticin (G418, Gibco-BRL). The cells were also plated on 4-well chamber slides at a density of 2×10⁵ cells/well.

Fluorescence confocal microscope. Human macrophages or FPRL1/293 cells grown on chamber slides were treated with FPRL1 agonists for different periods at 37° C. The cells were fixed in 4% paraformaldehyde for 10 min at room temperature. Slides were washed with PBS and incubated with 5% normal goat serum (Sigma) in PBS, 0.05% Tween-20 (PBS-T-NGS), for 1 h to block nonspecific binding sites and for permeabilization. The anti-Aβ₄₂ and anti-FPRL1 antibodies were applied and the slides were incubated for 1 h at room temperature. After three rinses with PBS, the slides were incubated with a mixture of FITC-conjugated goat anti-rabbit IgG and rhodamine-conjugated goat anti-mouse IgG (Sigma, 1:150 in TBS containing 3% BSA) for 30 min. The slides were mounted with an anti-fade, water-based mounting medium with 4,6-diamidino-2-phenylindole (DAPI; Vector Lab, Burlingame, Calif.) and analyzed under a laser scanning confocal fluorescence microscope (Leica TCS-4D DMIRBE, Heidelberg, Germany). Excitation wavelengths of 365 (for DAPI), 488 (for FITC), and 568 (for rhodamine) nm were used to generate fluorescence emission in blue, green, and red respectively. Colocalization of FPRL1 (green) and Aβ₄₂ (red) was reflected by yellow.

Congo red histochemistry. Cells on chamber slides were fixed with 4% paraformaldehyde and stained with hematoxylin for 2 min at room temperature. After 20 min incubation in a saturated NaCl solution containing 80% ethanol and 0.1% NaOH, the slides were reacted for 20 min with 0.2% Congo red. Destaining and dehydration were completed by washing the slides sequentially in 95% ethanol and 100% ethanol, followed by xylene. Coverslips were applied using Permount and the slides were viewed under light microscopy.

Detection of apoptosis. Apoptotic cells were detected by double labeling with annexin-V-FITC and propidium iodide (P1). Annexin-V binds to phosphatidylserine residues, which are translocated from the inner to the outer leaflet of the plasma membrane during the early stages of apoptosis (Koopman G. et al. 1994 Blood 84:1415-1420; Martin S. J. et al. 1995 J Exp Med 182:1545-1556). Necrotic cells were distinguished from annexin-V-positive cells by counterstaining with PI (final concentration 1 μg/ml) (Heidenreich S. et al. 1997 J Immunol 159:3178-3188; Mangan D. F. et al. 1991 J Immunol 146:1541-1546. Apoptotic cells were labeled by using an annexin-V kit according to manufacturer's instructions (Santa Cruz Biotechnology, Santa Cruz, Calif.) and analyzed by flow cytometry.

Internalization of FPRL1 induced by the agonist W pep. In permeabilized FPRL1/293 cells, FPRL1 was detected with a polyclonal antibody and was distributed mostly on the cell membrane region, as detected by fluorescence with confocal microscopy. Therefore, we first studied the localization and trafficking of FPRL1 after incubation with W pep, which is derived from a random peptide library and is a highly potent chemotactic agonist for FPRL1 (Le Y. et al. 1999 J Immunol 163:6777-6784; Seo J. K. et al. 1998 Clin Biochem 31:137-141). W pep dose-dependently induced a rapid internalization of FPRL1, which reached maximum after 15-30 min treatment at 37° C., with most of the fluorescence localized in the cytoplasmic compartment of the FPRL1/293 cells. When W pep was removed from culture medium after 30 min incubation with the cells, the fluorescence progressively intensified on the membrane region; after 2 h, most of the fluorescence was located on the cell surface. These results indicate that agonist-induced internalization of FPRL1 and receptor recycling after removal of the agonist can be detected by confocal microscopy.

Colocalization of Aβ₄₂ and FPRL1. As our study had revealed that Aβ₄₂ is a chemotactic agonist for FPRL1 (see Examples 1 and 2), we investigated the capacity of Aβ₄₂ to induce FPRL1 internalization. Incubation of FPRL1/293 cells and human macrophages for 30 min with Aβ₄₂ induced the internalization of FPRL1 in association with Aβ₄₂ into the cytoplasmic compartment in a dose-dependent manner. Maximal internalization of Aβ₄₂ and FPRL1 complexes occurred when 10 μM or more Aβ₄₂ was used to stimulate the cells. Similar concentrations of Aβ₄₂ have been shown to induce potent chemotaxis and Ca²⁺ flux in mononuclear phagocytes (monocytes and microglia) and FPRL1/293 cells as described above (see Examples 1 and 2). The concentrations of Aβ₄₂ used in our study were within or below those used by other laboratories to study the biological activities of the Aβ₄₂ (Hartley D. M. et al. 1999 J Neurosci 19:8876-8884; London J. A. et al. 1996 PNAS USA 93:4147-4152; McDonald D. R. et al. 1998 J Neurosci 18:4451-4460; Lorton D. et al. 2000 Neurobiol Aging 21:463-473; Combs C. K. et al. 1999 J Neurosci 19:928-939). Such concentrations of Aβ₄₂ have been detected in brain tissues of AD patients and mice transfected with human amyloid precursor protein gene (Kuo Y. M. et al. 1999 Biochem Biophys Res Commun 257:787-791; McLean C. A. et al. 1999 Ann Neurol 46:860-866; Kawarabayashi T. et al. 2001 J Neurosci 21:372-381; Funato H. et al. 1998 Am J Pathol 152:1633-1640) and are pathophysiologically relevant. In control experiments, neither Aβ₄₂ nor FPRL1 was detected in parental HEK293 cells even after treatment with 20 μM of Aβ₄₂. Investigation of the kinetics showed that at 5 min, Aβ₄₂ and FPRL1 were colocalized on the cell surface, followed by a rapid and progressive internalization of the Aβ₄₂/FPRL1 complex. As for W pep, the Aβ₄₂-induced FRPL1 internalization reached a maximal level at 15-30 min in FPRL1/293 cells and macrophages. When FPRL1/293 cells or macrophages were further cultured in Aβ₄₂-free medium, the FPRL1 could be detected on the cell surface within 2 h, suggesting rapid receptor recycling after depletion of Aβ₄₂ from culture supernatant. However, the antigenic Aβ₄₂ was detectable in the cytoplasmic region even 24 h after removal of Aβ₄₂. These data indicate that a transient interaction of Aβ₄₂ with FPRL1 promotes internalization of the ligand/receptor complex and that Aβ₄₂ was released intracellularly before the receptor FPRL1 travels back to the cell surface.

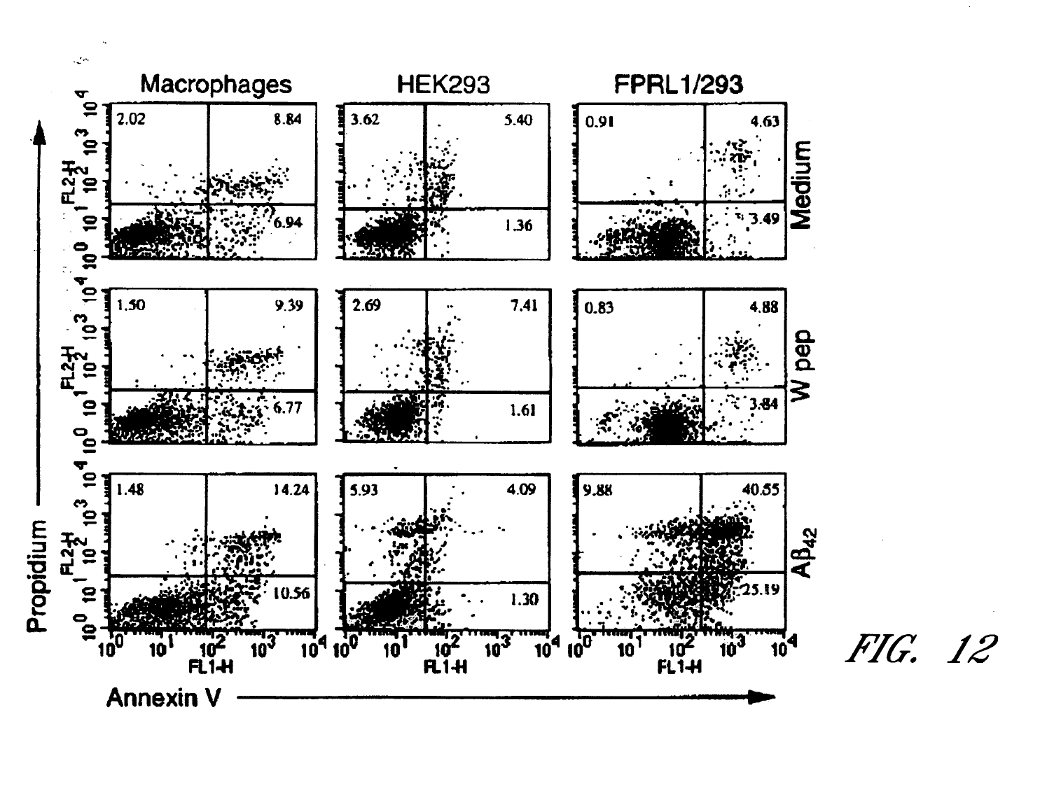

The effect of persistent exposure of FPRL1 to Aβ₄₂. Since a hallmark of AD is an aberrant and continual production and deposition of Aβ₄₂ in the brain, we investigated the effect of prolonged treatment of FPRL1/293 cells and macrophages with Aβ₄₂ on FPRL1 internalization and recycling. The persistent presence of Aβ₄₂ in culture supernatant for up to 48 h resulted in the retention of Aβ₄₂/FPRL1 complexes in the cytoplasmic region in FPRL1/293 cells and macrophages, and no FPRL1 could be detected on the cell surface. A cytopathic effect was observed when macrophages or FPRL1/293 cells were exposed to Aβ₄₂ for 48 h as shown by increased proportion of apoptotic cells (FIG. 12). In contrast to Aβ₄₂, W pep treatment for 48 h did not increase the apoptosis of FPRL1/293 cells or macrophages. Aβ₄₂ did not induce any apoptosis of parental HEK293 cells. These results indicate that the apoptotic effect was specific for Aβ₄₂ through its interaction with FPRL1.

Formation of fibrils in macrophages exposed to Aβ₄₂. It is well known that Aβ₄₂ forms fibrillar aggregates both in vivo and in vitro, so we investigated the effect of Aβ₄₂/FPRL1 internalization on intracellular aggregation of Aβ₄₂. Macrophages incubated with Aβ₄₂ for 24 h were stained positively with Congo red; this staining was markedly intensified at 48 h, suggesting that when Aβ₄₂ is internalized with FPRL1 in macrophages, it has the potential to become aggregated. Although massive colocalization of Aβ₄₂/FPRL1 could be observed at 24 h and 48 h in FPRL/293 cells, we failed to detect Congo red positive fibrils in these cells. These results indicate that Aβ₄₂ internalized into FPRL1 transfected cells do not undergo intracellular fibrillar formation as in macrophages. However, these cells exhibited a greater tendency than macrophages to undergo apoptotic death after exposure to Aβ₄₂ (FIG. 12). In contrast to Aβ₄₂, W pep did not form Congo red positive fibrils in macrophages after 48 h incubation, indicating that even though Aβ₄₂ and W peptide are both agonists for FPRL1, they exhibited very different physicochemical properties.

Effect of colchicine on Aβ₄₂/FPRL1 internalization. Having established that Aβ₄₂ associated with FPRL1 could be rapidly internalized and the internalized Aβ₄₂ form fibrils in macrophages, we asked whether anti-inflammatory agents might interfere with the interaction between Aβ₄₂ and FPRL1 and the subsequent ligand/receptor internalization. We used colchicine, an antimitotic agent that has been reported to inhibit the function of microtubules (Rossi M. et al. 1996 Biochemistry 35:3286-3289) and to abolish Aβ₄₂-induced monocyte release of neurotoxic mediators (Dzenko K. A. et al. 1997 J Neuroimmunol 80:6-12; Heinzelmann M. et al. 1999 J Immunol 162:4240-4245). We observed that colchicine was a potent inhibitor of Aβ₄₂-induced chemotaxis of both monocytes and FPRL1/293 cells. In macrophages and FPRL1/293 cells treated with colchicine, Aβ₄₂ still rapidly associated and could be colocalized with FPRL1 on the cell surface within 5 min. However, the Aβ₄₂/FPRL1 complexes failed to internalize and remained on the cell surface even after 30 min incubation at 37° C. These results indicate that whereas colchicine does not inhibit the cell surface expression of FPRL1 and its initial binding of Aβ₄₂, it interferes with FPRL1/Aβ₄₂ complex internalization and the resultant cell signaling (chemotaxis), presumably through inhibition of microtubule movement. Colchicine-treated macrophages were not significantly stained with Congo red after 48 h incubation with Aβ₄₂: only a brownish staining was visible, which could have been due to early stage of extracellular aggregation of Aβ₄₂. Thus, colchicine appears to be capable of preventing cell activation by Aβ₄₂ and the subsequent intracellular deposition of Aβ₄₂ fibrils in macrophages.

Although the invention has been described with reference to embodiments and examples, it should be understood that various modifications can be made without departing from the spirit of the invention. Accordingly, the invention is limited only by the following claims. All references cited herein are hereby expressly incorporated by reference.

1. An isolated Aβ FPR class receptor complex.

2. A method of identifying an agent that modulates the assembly of an Aβ-FPR class receptor complex comprising the steps of:
providing a support having disposed thereon at least one molecule of Aβ or an FPR class receptor;
contacting said support with at least one molecule of an FPR class receptor when said support has disposed thereon at least one molecule of Aβ, or at least one molecule of Aβ when said support has disposed thereon at least one molecule of an FPR class receptor, in the presence and absence of at least one test material; and
identifying said test material as an agent that modulates the assembly of said Aβ-FPR class receptor complex if said test material alters the association of said Aβ and said FPR class receptor to form said Aβ-FPR class receptor complex.

3. A method of identifying an agent that modulates the assembly of an Aβ-FPR class receptor complex comprising the steps of:
providing a support having disposed thereon an Aβ-FPR class receptor complex;
contacting said support in the presence of at least one test material; and
identifying said test material as an agent that modulates the assembly of said Aβ-FPR class receptor complex, wherein said test material alters the association of Aβ and an FPR class receptor to form an Aβ-FPR class receptor complex.

4. A method of modulating an Aβ-FPR-class receptor complex-mediated inflammatory response in a subject comprising the steps of:
identifying a subject in need thereof; and
administering to said subject an agent that modulates assembly of said Aβ-FPR class receptor complex.

5. The method of claim 4, wherein said subject has Alzheimer's disease.

6. The method of claim 4, further comprising measuring the effect of said agent on the assembly of said Aβ-FPR class receptor complex.

7. A method of identifying an agent that modulates the signal transduction mediated by the assembly of an Aβ-FPR class receptor complex comprising the steps of:
providing a support having disposed thereon at least one molecule of Aβ or an FPR class receptor;
contacting said support with at least one molecule of an FPR class receptor when said support has disposed thereon at least one molecule of Aβ, or at least one molecule of Aβ when said support has disposed thereon at least one molecule of an FPR class receptor, in the presence of at least one test material; and
identifying said at least one test material as an agent that modulates the signal transduction mediated by the assembly of said Aβ-FPR class receptor complex, wherein said at least one test material alters the signal transduction generated by the association of Aβ and an FPR class receptor to form an Aβ-FPR class receptor complex.

8. A method of identifying an agent that modulates the signal transduction mediated by the assembly of an Aβ-FPR class receptor complex comprising the steps of:
providing a support having disposed thereon an Aβ-FPR class receptor complex;
contacting said support in the presence of at least one test material; and
identifying said at least one test material as an agent that modulates the signal transduction mediated by the assembly of said Aβ-FPR class receptor complex, wherein said at least one test material alters the signal transduction generated by the association of Aβ and an FPR class receptor to form an Aβ-FPR class receptor complex.

9. A method of making a pharmaceutical comprising:
identifying an agent that modulates the assembly of an Aβ-FPR class receptor complex according to the method of claim 2 or 3; and
incorporating a therapeutically effective amount of said agent into a pharmaceutical.

10. A method of making a pharmaceutical comprising:
identifying an agent that modulates the signal transduction mediated by the assembly of an Aβ-FPR class receptor complex according to the method of claim 7 or 8; and
incorporating a therapeutically effective amount of said agent into a pharmaceutical.

11. The method of claim 2, 3, 7, or 8, wherein the FPR class receptor is FPR or FPRL1.
